SEQUENCE LISTING (28 sequences)

SEQ ID NO: 1 - DNA, 3074 bp, Homo sapiens
agagacttcagaccctaattgctctcctcagggcgcccgaggaggcaagctggggcagggatccctttagccacccagaccgaatctcggagggtacctgactgcgggcggccagtccacagccccctcccgccgccccggccggagaagaggcaggttctgcgcagagctgccaccctcgccagcgtcgccagcatcgcgtgcgtctcagatggcatcggtcccggtgcaagcggcgaactccgtgcggcgccacccgccgccactggggggcgcccacggacgccgcagaccccgcccccgccgggcggggctgagcactagtcgcctgctgggccagcttctagtctgtcggtcagaccaagggcactcagactggaaaggttgttgctgggcttgggtgtccgtcctttcctgtctccttttcttcctcttttcacttaaatccactgagtctctcgccaagtcagtgatttaggtaatcccgccgcccctggcggaacctttgtgacgccggaactgatgtgccgtagggtggctgcggtcggaggactgttttggtaaaggactagcagttctctgcggagggccggttgatacagttccggtgggagaacgcggctgcgaggttttcggctttggctcctgatatgcagcgacagaattttcggcccccaactcctccttaccctggtccgggtggaggaggttggggtagcggaagcagcttccggggaaccccgggcgggggcggaccacggccgccctcccctcgagacgggtacgggagtccgcaccacacgccgccgtacgggccccggtctaggccgtacgggagcagtcactctccgcgacacggcggcagcttcccggggggccggttcgggtctccgtcccctggcggctaccctggctcctactccaggtcccccgcggggtcccagcagcaattcggctactccccagggcagcagcagacccacccccaggtaataataatagccagcgtttgccgagctcttactttatggcaggcacggtactaatattccctttacgtggattattttgtctaatcctctctgaggtactattgttagcccaattttgcagatgagcgaactgaggcttagagaagttgagtgacttccggaagattatccgaaagagtggcagagccgtacgttctgttttcgagccctcgtgcttttaaccaatacactgccacccgtacacagattttcctaattctccatttcctcctgtttgccaaaggccttgccagtttgtttcaagaaatagttctttttcttttttttttttttttttttatctttctttcgagctacactggcttctgctccaacttccctgaaaagaaaatcggtggtagtggaagttttccattcctttagtgagagtaaatacaaaagacagagagagtatctctagcatgtctgggatttctgtggtttacaacttgcaactacgtggttttccaacttgctgtattctacaccaaccccctaccccttttgagcctggagctaaggagtaaagaaaatgttcatgagctagaaaggttctagaaactttaaaactatagtattgttttctcctgaaaaaagtgaaaatttcgagggaaaattgaatataattacttgatttcatcactgcttgtatttaattactgcaaagttactacattagacacaaaagtatttgagatgacagtggtcatggaattataaataaatgagaataacgaattgctttaagtactttttttaaaaaataggaacacttgattaaaggaagcatggactttgtaattgaacattatttaggttcaagtcacaacttttaagcagcaatgtgattcccgctaacctttagctaaaggaagaagtaccaattatctttactaagtgtttttttaactgattttttttctaaagggttctccaaggacatctacaccatttggatcagggcgtgttagagaaaaaagaatgtctaatgagttggaaaattatttcaagccttcaatgcttgaagatccttgggctggcctagaaccagtatctgtagtggatataagccaacaatacagcaatactcaaacattcacaggcaaaaaaggaagatacttttgttaacatttctgaaattcaactggaagcttcatgtgtcaggaacatcttggacaaaactttaagttgtgttgatataaatttacccaaagatgatgactttgattggataattagtaaggtctttttgttatttttcatcgtatcaggtattgttgatattagagaaaaaagtaggataacttgcaacatttagctctggaagtacctaccacattttagagatttaccgtttccatatatttaacattcctggttacataatggacatttgtcttttaatgttttttcaatgttttaaaataaaacattttgtcttctagctattgtggttttgtggtatgataaagaagtagacttactacagtaatgctttgtagtcacttagagttcataggtaaatgttttgcaaattatttttgaaaatgaaataggtaaaccatcctttgagctgtagacagctctgtatttgttcgtaaataatgaggatgagaataacagagttctgttcatactcattttatctaatgcttctgttaaaatgcattaaaacttgaagccccagaaacccatttaagcataactttatactcatttaactggaaaaggcaagaatagaccatgcattgctaaatcaaagtgttctatctgaatttctctatacctcttagcattatggagttagtttctcttgaggccaaagaagatagctaccagcagcctcaaatcacttccattgcaaaaagaacatctctcatctccatcaatccaagagaagacaaatttgccatacttagatcatgtttctgtattattgaaccaattaactgtggccaaggccacagtgagacctaattaacttgaatcatggacacatcaccataatgagacaggattctgtgaccttgaacaccactgggc

SEQ ID NO: 2 - DNA, 540 bp, Homo sapiens
atgcagcgacagaattttcggcccccaactcctccttaccctggtccgggtggaggaggttggggtagcggaagcagcttccggggaaccccgggcgggggcggaccacggccgccctcccctcgagacgggtacgggagtccgcaccacacgccgccgtacgggccccggtctaggccgtacgggagcagtcactctccgcgacacggcggcagcttcccggggggccggttcgggtctccgtcccctggcggctaccctggctcctactccaggtcccccgcggggtcccagcagcaattcggctactccccagggcagcagcagacccacccccagggttctccaaggacatctacaccatttggatcagggcgtgttagagaaaaaagaatgtctaatgagttggaaaattatttcaagccttcaatgcttgaagatccttgggctggcctagaaccagtatctgtagtggatataagccaacaatacagcaatactcaaacattcacaggcaaaaaaggaagatacttttgttaa

SEQ ID NO: 3 - protein, 178 aa, Homo sapiens
MQRQNFRPPTPPYPGPGGGGWGSGSSFRGTPGGGGPRPPSPRDGYGSPHHTPPYGPRSRPYGSSHSPRHGGSFPGGRFGSPSPGGYPGSYSRSPAGSQQQFGYSPGQQQTHPQGSPRTSTPFGSGRVREKRMSNELENYFKPSMLEDPWAGLEPVSVVISQQYSNTQTFTGKKGRYFC

SEQ ID NO: 4 - DNA, 19 bp, Artificial
  Synthetic Primer
gtctcagatggcatcggtc

SEQ ID NO: 5 - DNA, 20 bp, Artificial
  Synthetic Primer
gttctcccaccggaactgta

SEQ ID NO: 6 - DNA, 20 bp, Artificial
  Synthetic Primer
gaactgatgtgccgtagggt

SEQ ID NO: 7 - DNA, 20 bp, Artificial
  Synthetic Primer
aagtaagagctcggcaaacg

SEQ ID NO: 8 - DNA, 20 bp, Artificial
  Synthetic Primer
caatgtgattcccgctaacc

SEQ ID NO: 9 - DNA, 23 bp, Artificial
  Synthetic Primer
tcataccacaaaaccacaatagc

SEQ ID NO: 10 - DNA, 28 bp, Artificial
  Synthetic Primer
gactttggaaaattatttcaagccttca

SEQ ID NO: 11 - DNA, 22 bp, Artificial
  Synthetic Primer
tgtggactcccttgctaagaat

SEQ ID NO: 12 - DNA, 22 bp, Artificial
  Synthetic Primer
gaaaaacccaatcaccaaaatg

SEQ ID NO: 13 - DNA, 20 bp, Artificial
  Synthetic Primer
ggcgagagactcagtggatt

SEQ ID NO: 14 - DNA, 20 bp, Artificial
  Synthetic Primer
atccctttagccacccagac

SEQ ID NO: 15 - DNA, 20 bp, Artificial
  Synthetic Primer
cgtttgccgagctcttactt

SEQ ID NO: 16 - DNA, 20 bp, Artificial
  Synthetic Primer
gcaagttggaaaaccacgta

SEQ ID NO: 17 - DNA, 24 bp, Artificial
  Synthetic Primer
ggttcaagtcacaacttttaagca

SEQ ID NO: 18 - DNA, 23 bp, Artificial
  Synthetic Primer
tcaaagtcatcatctttgggtaa

SEQ ID NO: 19 - DNA, 20 bp, Artificial
  Synthetic Primer
tggccatttggtttgttacc

SEQ ID NO: 20 - DNA, 21 bp, Artificial
  Synthetic Primer
gcccctataaggagaccctct

SEQ ID NO: 21 - DNA, 20 bp, Artificial
  Synthetic Primer
ccactcacacatccatgtcc

SEQ ID NO: 22 - DNA, 20 bp, Artificial
  Synthetic Primer
caaacaaaagccaaagcaaa

SEQ ID NO: 23 - DNA, 20 bp, Artificial
  Synthetic Primer
tcctttcttgcaggcttgat

SEQ ID NO: 24 - DNA, 20 bp, Artificial
  Synthetic Primer
ttctgaaagagccagccagt

SEQ ID NO: 25 - DNA, 20 bp, Artificial
  Synthetic Primer
gagtgaaggcagccctgtta

SEQ ID NO: 26 - DNA, 20 bp, Artificial
  Synthetic Primer
aaaaggcagaaggctgaggt

SEQ ID NO: 27 - DNA, 20 bp, Artificial
  Synthetic Primer
agccaggccagagaacacta

SEQ ID NO: 28 - DNA, 20 bp, Artificial
  Synthetic Primer
gggtcctccctctagcctta

(19) United States
(12) Patent Application Publication
(10) Pub. No.: US 2006/0172322 A1
(43) Pub. Date: Aug. 3, 2006

(54) Mutations in the C7orf11 (TTDN1) gene causative of non-photosensitive trichothiodystrophy

(76) Inventors: Kazuhiko Nakabayashi, Toronto (CA); Stephen W. Scherer, Toronto (CA)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/261,085

(22) Filed: Oct. 28, 2005

Related U.S. Application Data
(60) Provisional application No. 60/622,597, filed on Oct. 28, 2004.

(51) Int. Cl. C12Q 1/68; C07H 21/04; C12P 21/06; C12N 5/06; C12N 15/74; C12N 1/18; C12N 5/04; C07K 14/705

(52) U.S. Cl. 435/6; 435/69.1; 435/320.1; 435/325; 530/350; 536/23.5; 435/252.33; 435/254.2; 435/348; 435/419

(57) ABSTRACT

The invention is the demonstration that mutations of the C7orf11 nucleic acid sequence leads to the development of non-photosensitive trichothiodystrophy. The invention comprises methods of screening for and detection of non-photosensitive TTD carriers, prenatal screening and diagnosis, diagnosis of non-photosensitive TTD, drug and gene therapy, manufacture of the protein, and antibodies as well as development of animal models of disease for the testing of pharmaceutical agents.

### FIELD OF THE INVENTION

The invention relates generally to the identification of the C7orf11 gene as causative of non-photosensitive trichothiodystrophy (TTD). More specifically, the invention is the identification, isolation and cloning of the C7orf11 DNA sequence corresponding to normal and mutant forms of the gene as well as their transcripts and gene products. Mutation of the C7orf11 DNA sequence is demonstrated to lead to the development of non-photosensitive trichothiodystrophy. The invention relates to methods of screening for and detection of non-photosensitive TTD carriers, prenatal screening and diagnosis, diagnosis of non-photosensitive TTD, drug and gene therapy, manufacture of the protein as well as development of animal models of disease for the testing of pharmaceutical agents.

### BACKGROUND OF THE INVENTION

Trichothiodystrophy (TTD), or sulfur-deficient brittle hair¹, is a congenital disorder that can be associated with a spectrum of symptoms affecting organs derived from ectodermal and neuroectodermal origin (Table 4). Typically TTD is marked by abnormal sulfur-deficient brittle hair that is dry, sparse and easily broken. Other symptoms include nail dystrophy, mental and growth retardation, ichthyosis (scaly, fish-like skin), decreased fertility, infertility and cutaneous photosensitivity that is not associated with a predisposition towards the development of cancer². Approximately half of TTD patients display photosensitivity. All of these symptoms are associated with defects in nucleotide excision repair (NER) due to mutations the subunits of TFIIH XPD3, XPB4, or TTD-A5 that cause a reduction of cellular concentration of transcription factor IIH (TFIIH)6. TFIIH is a protein complex of nine subunits which was originally identified as an essential factor in basal transcription initiation. TFIIH is required for local unwinding of the DNA helix around the lesion in NER and in the transcription initiation of RNA polymerase II at the promoter.

Amish brittle hair syndrome (ABHS) (OMIM:234050), is an autosomal recessive disorder characterized by short stature, intellectual impairment, sulfur-deficient brittle hair, and decreased male fertility, but no cutaneous photosensitivity^{7,8}. Other forms of non-photosensitive TTD such as Sabinas⁹ and Pollitt¹⁰ syndromes are hypothesized to be allelic with ABHS because of similar clinical presentation.

The application of genetic and molecular cloning strategies has now demonstrated that the C7orf11 gene is a causal gene for non-photosensitive TTD.

### SUMMARY OF THE INVENTION

The gene involved in non-photosensitive TTD has now been identified to be the C7orf11 gene (herein also referred to as “TTDN1”). Mutations in the gene sequence leads to the development of non-photosensitive TTD in a subgroup of non-photosensitive TTD patients. Mutations were found in Amish brittle hair syndrome and other non-photosensitive TTD cases exhibiting symptoms such as but not limited to mental retardation, decreased fertility, infertility but not in Sabinas or Pollitt syndrome.

The gene involved in non-photosensitive TTD and its functional equivalents, has been identified as the C7orf11 gene. The C7orf11 gene has been isolated and cDNA cloned, and its transcripts and gene products have been identified and sequenced. Mutations in the C7orf11 gene are demonstrated to be causative of non-photosensitive TTD. A homozygous A>G DNA sequence variant leads to an amino acid substitution M144V in the gene product. Furthermore, a 2 bp homozygous deletion in Exon 1 has been found to create a predicted 57 amino acid truncated protein. Further, parts of Exon 1 and/or Exon 2 or all of Exon 1 and Exon 2 may be homozygously deleted in which case the patient is genetically null for the C7orf11 gene. Lastly, the absence of the entire C7orf11 gene will also lead to non-photosensitive TTD.

With the identification and sequencing of the gene and its gene product, nucleic acid probes and antibodies raised to the gene product can be used in a variety of hybridization and immunological assays to screen for and detect the presence of either a normal or a mutated C7orf11 gene or gene product in a subject such as a human. Assays may in general also be used to detect the complete absence of the C7orf11 gene. Assay kits for such screening and diagnosis can also be provided.

Patient therapy through supplementation with the normal gene product, whose production can be amplified using genetic and recombinant techniques, or its functional equivalent, is now also possible. Correction or modification of the defective gene product through drug treatment means is embodied. In addition, non-photosensitive TTD may be treated or controlled through gene therapy by correcting the gene defect in situ or using recombinant or other vehicles to deliver a DNA sequence capable of expression of the normal gene product to the cells of the patient.

Animal models of disease can now be developed in order to study the mechanism of the function of the C7orf11 gene and also allow for the screening of possible therapeutic compositions to help alleviate, treat and/or prevent disease.

According to an aspect of the invention, there is provided a human gene isolated from human chromosome 7p14, said gene comprising a cDNA sequence having exons 1 to 2 which encodes C7orf11 protein, wherein mutations in said gene are causative of non-photosensitive TTD.

The genomic sequence for the C7orf11 gene is shown in Table 1 as Sequence ID No. 1. The cDNA sequence is shown in Table 2 represented as Sequence ID No. 2. The amino acid sequence of the C7orf11 protein is shown in Table 3 and represented by Sequence ID No. 3.

According to an aspect of the invention is a nucleic acid sequence encoding a C7orf11 protein, said nucleic acid sequence having a mutation leading to the development of non-photosensitive Trichothiodystrophy (TTD) in a subject.

According to another aspect of the invention, a nucleic acid sequence comprises a sequence selected from the group consisting of:

(a) nucleic acid sequences which correspond to a fragment of the sequence of Sequence ID No. 1 or Sequence ID No. 2;

(b) nucleic acid sequences which comprise at least 18 nucleotides and encode a fragment of the amino acid sequence of Sequence ID No. 3; and

(c) nucleic acid sequences encoding an epitope encoded by at least 18 sequential nucleotides in the sequence of Sequence ID No. 1 or Sequence ID No. 2.

According to a further aspect of the invention is a purified nucleotide sequence wherein said sequence may comprise genomic DNA, cDNA, antisense DNA, homologous DNA, or mRNA, and is selected from at least one of the exons of the cDNA sequence of SEQ ID No: 2, shown in Table 2.

According to another aspect of the invention, a purified mutant C7orf11 gene comprises a DNA sequence encoding an amino acid sequence for a protein where the protein, when expressed in cells of the human body, is associated with a symptom selected from the group consisting of brittle hair, mental retardation, decreased fertility, ichthyosis and combinations thereof, wherein the symptom correlates with the genetic disease non-photosensitive trichothiodystrophy.

According to another aspect of the invention, a DNA molecule comprises a DNA sequence encoding mutant C7orf11 polypeptide having the sequence shown in Table 3 for amino acid residue positions 1 to 179. The sequence is further characterized by a mutation selected from the group consisting of M144V, a truncated polypeptide and deletion of one or both of exons 1 and 2 and portions thereof.

A purified nucleic acid probe comprising a DNA or RNA nucleotide sequence corresponding to any mutant sequences as recited above.

According to another aspect of the invention, a recombinant cloning vector comprising the nucleic acid sequences of the normal or mutant C7orf11 DNA and fragments thereof is provided. The vector, according to an aspect of this invention, is operatively linked to an expression control sequence in the recombinant DNA molecule so that the normal C7orf11 protein can be expressed, or alternatively with the other selected mutant nucleic acid sequence the mutant C7orf11 polypeptide can be expressed. The expression control sequence is selected from the group consisting of sequences that control the expression of genes of prokaryotic or eukaryotic cells and their viruses and combinations thereof.

According to another aspect of the present invention is a method for screening a subject to determine if said subject is a carrier of a mutant C7orf11 gene leading to the development or diagnosis of non-photosensitive TTD, said method comprising:

determining from a biological sample from said subject the presence of a deletion mutation, point mutation, frame-shift mutation or a null mutation of the C7orf11 gene.

In aspects, the determination of the entire absence of the gene is indicative of

According to another aspect of the present invention is a method for screening a subject to determine if said subject is a carrier of a mutant form of the C7orf11 gene leading to the development or diagnosis of non-photosensitive TTD in said subject, the method comprising:

(a) assaying a biological sample of a subject to be screened, said sample containing a mutant or a normal C7orf11 gene, wherein the assay comprises:

(i) assaying for the presence of a normal C7orf11 gene by hybridization comprising: (A) an oligonucleotide probe which specifically binds to a normal nucleic acid sequence encoding a normal C7orf11 polypeptide, wherein the normal DNA molecule comprises a nucleic acid sequence selected from the group consisting of:

(1) a nucleic acid sequence encoding a normal C7orf11 protein having the amino acid sequence shown in Table 3;

(2) a nucleic acid sequence which hybridizes under stringent conditions to at least 16 contiguous nucleotides of the nucleic acid sequence of (1); and

(3) a nucleic acid sequence complementary to the nucleic acid sequence of (1) or (2), and

(B) providing at least one reagent for detecting the hybridization of the oligonucleotide probe to said normal nucleic acid sequence; or

(ii) assaying for the presence of a mutant C7orf11 gene by hybridization comprising:

(A) an oligonucleotide probe which specifically binds to a mutant nucleic acid sequence encoding a mutant C7orf11 polypeptide, wherein the mutant nucleic acid sequence comprises a nucleic acid sequence selected from the group consisting of:

(1) a nucleic acid sequence encoding a mutant C7orf11 protein having a M144V amino acid substitution in the amino acid sequence depicted in Table 3;

(2) a nucleic acid sequence which hybridizes under stringent conditions to at least 16 contiguous nucleotides of the nucleic acid sequence of (1), said nucleic acid sequence containing said amino acid substitution; and

(3) a nucleic acid sequence complementary to the nucleic acid sequence of (1) or (2); and

(B) providing at least one reagent for detecting the hybridization of the oligonucleotide probe to said mutant nucleic acid sequence,

wherein the probe and the reagent in (i) and (ii) are each present in amounts effective to perform the hybridization assay.

According to another aspect of the present invention is a kit for assaying the presence of a normal C7orf11 gene by hybridization, said kit comprising:

(a) an oligonucleotide probe which binds to a normal nucleic acid sequence selected from the group consisting of:

(i) a nucleic acid sequence encoding a normal C7orf11 protein having the amino acid sequence depicted in Table 3;

(ii) a nucleic acid sequence which hybridizes under stringent conditions to at least 16 contiguous nucleotides of the nucleic acid sequence of (i); and

(iii) a nucleic acid sequence complementary to the nucleic acid sequence of (i) or (ii); and

(b) at least one reagent for detecting the hybridization of the oligonucleotide probe to said nucleic acid sequence, wherein the probe and the reagent are each present in amounts effective to perform the hybridization assay.

According to another aspect of the present invention is a kit for assaying the presence of a mutant C7orf11 gene by hybridization, said kit comprising:

(a) an oligonucleotide probe which specifically binds to a mutant nucleic acid sequence comprising a nucleic acid sequence selected from the group consisting of:

(i) a nucleic acid sequence encoding a mutant C7orf11 protein having an amino acid substitution M144V depicted in Table 3;

(ii) a nucleic acid sequence that contains a deletion in one or both of exons 1 and 2 as depicted in Table 2;

(iii) a nucleic acid sequence which hybridizes under stringent conditions to at least 16 contiguous nucleotides of the DNA sequence of (i) or (ii), said nucleic acid sequence containing the amino acid substitution; and

(iv) a nucleic acid sequence complementary to the nucleic acid sequence of (i), (ii) or (iii); and

(b) at least one reagent for detecting the hybridization of the oligonucleotide probe to said nucleic acid sequence,

wherein the probe and the reagent are each present in amounts effective to perform the hybridization assay.

According to another aspect of the invention, a method is provided for screening a subject to determine if the subject is a carrier of a mutant C7orf11 gene, said method comprising the steps of providing a biological sample of the subject to be screened and providing an assay for detecting in the biological sample, the presence of one or more mutations and/or deletions in the C7orf11 gene sequence. Such mutations and/or deletions being indicative of being a non-photosensitive TTD carrier.

According to another aspect of the invention, a method is provided for screening a subject to determine if the subject is a carrier of a mutant C7orf11 gene comprising the steps of providing a biological sample of the subject to be screened and providing an assay for detecting in the biological sample, the presence of at least a member from the group consisting of the normal C7orf11 gene, normal C7orf11 gene products, a mutant C7orf11 gene, mutant C7orf11 gene products and mixtures thereof. In aspects, the subject may be a fetus and detection of a heterozygous mutation indicating the fetus is a carrier for non-photosensitive TTD. A homozygous mutation indicating that the fetus may after birth develop a symptom selected from the group consisting of brittle hair, nail dystrophy, mental retardation, growth retardation, ichthyosis, decreased fertility, infertility and combinations thereof. Such a homozygous mutation being indicative of the development of TTD and in particular, non-photosensitive TTD.

According to another aspect of the invention, a method is provided for the diagnosis of non-photosensitive TTD in a subject, the method comprising the steps of providing a biological sample of the subject to be screened and providing an assay for detecting in the biological sample the presence of one or more mutations and/or deletions in the C7orf11 gene sequence. Such mutations and/or deletions being indicative of being a non-photosensitive TTD carrier or afflicted with non-photosensitive TTD.

According to another aspect of the invention, a method is provided for the correlation of a symptom selected from the group consisting of mental retardation, decreased fertility or infertility with the disease of non-photosensitive TTD in a subject, the method comprising the steps of providing a biological sample of the subject to be screened and providing an assay for detecting in the biological sample the presence of one or more mutations and/or deletions in the C7orf11 gene sequence.

According to another aspect of the invention is a kit for identification of mutations in the C7orf11 gene, comprising a plurality of primer pairs for multiplex co-amplification of exons 1 and 2 of the gene, wherein one member of each primer pair is labeled with a detectable label, and wherein the primer pairs are selected to have a common melting temperature and to produce amplification products having differing lengths and wherein the primers pairs bind to intron regions immediately flanking each of the selected plurality of exons, said kit comprising at least one set of primers for amplification of the selected plurality of exons of the C7orf11 gene or cDNA sequence.

According to another aspect of the invention is a method for genetic screening of family members of an individual diagnosed as having non-photosensitive TTD, comprising the steps of:

(a) obtaining a patient blood sample from the diagnosed individual;

(b) quantitatively amplifying at least one exon of the C7orf11 gene in cells from the patient blood sample using primers complementary to intron regions immediately flanking each exon amplified; and

(c) determining the length of the amplification product for each exon amplified and comparing that length to the length of amplification products obtained when a wild-type C7orf11 gene is amplified using the same primers, whereby differences in length between an amplified sample exon and the corresponding amplified wild-type exon reflect the occurrence of an inherited deletion mutation in the C7orf11 gene of the diagnosed individual; and

(d) if an inherited mutation is identified, obtaining blood samples from the biological parents of the diagnosed individual; quantitatively amplifying the exon of the C7orf11 gene found to contain a deletion mutation in the patient blood sample in cells from the parent blood samples using the same primers used to amplify the exons in the patient blood sample; and determining the length of the amplification product for the exon in the amplified parent blood samples and comparing that length to the length of amplification products obtained when the patient blood sample was amplified, wherein in step (b) exons 1 and 2 are coamplified in a single reaction.

According to another aspect of the invention, an immunologically active anti-C7orf11 polyclonal or monoclonal antibody specific for the C7orf11 polypeptide is provided.

According to another aspect of the invention, a kit for assaying for the presence of a mutant C7orf11 gene by hybridization techniques comprising:

(a) an oligonucleotide probe which specifically binds to a mutant C7orf11 gene;

(b) reagent means for detecting the hybridization of the oligonucleotide probe to the C7orf11 gene; and

(c) the probe and reagent means each being present in amounts effective to perform the hybridization assay.

According to another aspect of the invention is a method for identifying mutations in a C7orf11 gene comprising the steps of:

(a) quantitatively co-amplifying the exons of the sample C7orf11 gene using primers complementary to intron regions immediately flanking each of the exons;

(b) determining the lengths of the amplification products for each amplified sample exon and comparing that length to the length of amplification products obtained when a wild-type C7orf11 gene is amplified using the same primers, whereby differences in length between an amplified sample exon and the corresponding amplified wild-type exon reflect the occurrence of a deletion mutation in the sample C7orf11 gene.

According to another aspect of the invention, is a kit for assaying for the presence of a mutant C7orf11 gene by immunoassay techniques comprises:

(a) an antibody which specifically binds to a mutant gene product of the C7orf11 gene;

(b) reagent means for detecting the binding of the antibody to the gene product; and

(c) the antibody and reagent means each being present in amounts effective to perform the immunoassay.

According to another aspect of the present invention is a method of producing a mutant mammalian C7orf11 polypeptide comprising:

providing a cell transformed with a nucleic acid sequence encoding a mutant C7orf11 polypeptide positioned for expression in said cell;

culturing said transformed cell under conditions for expressing said nucleic acid; and

producing said mammalian C7orf11 polypeptide.

In aspects of the invention, the mutant C7orf11 polypeptide contains one or more mutations and/or deletions in Seq ID No. 3. The mutant C7orf11 polypeptide is encoded by a nucleic acid sequence set forth in Seq ID No. 1 or 2, wherein such nucleic acid sequence contains one or more mutations and/or deletions in the sequences contained therein.

According to another aspect of the invention is an anti-C7orf11 polyclonal or monoclonal antibody specific for a normal C7orf11 polypeptide (SEQ ID NO:3).

According to another aspect of the invention is an anti-C7orf11 polyclonal or monoclonal antibody specific for a mutant C7orf11 polypeptide, wherein said amino acid sequence includes at least one mutation, wherein said mutation is an amino acid substitution M144V in the sequence of SEQ ID NO: 3.

According to another aspect of the invention is an anti-C7orf11 polyclonal or monoclonal antibody specific for a mutant C7orf11 polypeptide, wherein said mutant polypeptide is a truncated 57 amino acid polypeptide.

According to another aspect of the invention is a method is provided for treatment for non-photosensitive TTD in a patient. The treatment comprises the step of administering to the patient a therapeutically effective amount of the normal C7orf11 protein, or a functional fragment thereof, or a functional analogue thereof or a mimetic thereof.

According to another aspect of the invention, a method of gene therapy for non-photosensitive TTD in a patient comprises the step of delivery of a DNA molecule which includes a sequence corresponding to the normal DNA sequence encoding for normal C7orf11 protein.

According to another aspect of the invention, an animal comprises an heterologous cell system. The cell system includes a recombinant cloning vector which includes the recombinant DNA sequence corresponding to the mutant DNA sequence which induces non-photosensitive TTD symptoms in the animal.

According to another of inventions is a transgenic cell comprising a C7orf11 transgene having one or more mutations therein.

According to another aspect of the invention, a transgenic animal exhibits non-photosensitive TTD symptoms. In aspects, the transgenic animal is a mouse.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### BRIEF DESCRIPTION OF THE DRAWINGS

The present invention will become more fully understood from the detailed description given herein and from the accompanying drawings, which are given by way of illustration only and do not limit the intended scope of the invention.

FIG. 1 shows the identification of C7orf11 mutations. FIG. 1(a) shows a physical map of the 2 Mb 7p14 region showing homozygosity in a consanguineous Amish kindred⁷. FIG. 1(b) shows that C7orf11 consists of two exons spanning 2 kb. Coding and untranslated regions are shown in solid and open boxes, respectively. Red and blue asterisks indicate the position of the M144V mutation in the Amish kindred and the 2 bp deletion in Moroccan TTD siblings, respectively. Black and red bars (numbered from 1 to 11) represent the size and location of the PCR products in the deletion mapping for Pt. 6474 as shown in FIG. 1(e). The fragments that were not amplified from Pt. 6474 (due to homozygous deletion) are shown by red bars. FIG. 1(c) shows five affected families from the Amish kindred⁷. Solid (gray and black) and open squires/circles indicate affected and unaffected members, respectively. The member shown in black in Family B represents the proband, who had a more severe phenotype presumably due to a de novo chromosomal abnormality (46,XY,14q⁻)⁷. The genotype for the M144V mutation site of each member is shown (A, wild; G, mutated). FIG. 1(d) shows electropherograms for the M144V site. FIG. 1(e) shows representative results (by test primers #3, #4, #6, and #10) of the PCR-based deletion mapping for the C7orf11 locus on Pt. 6474. Each panel contains amplified products from a control (C) and Pt. 6474 (P). Solid rectangles indicate the 704 bp fragment amplified by control primers (DJ-g5/g6) in multiplex PCR. Open rectangle in each panel indicates the fragment amplified by a test primer pair (FIG. 1b).

FIG. 2(a) shows the human C7orf11 protein (top). The glycine/proline rich region is shown in green (low complexity regions detected by the BLASTP program is in light green). There are two highly conserved C terminal regions (CR1 and CR2) among candidate orthologues. The evolutionary tree (middle) was drawn by ClustalX (accession numbers are provided in the examples). Overall amino acid similarity with the human protein sequence, percentage ratio of glycine/proline content in the region from the N-terminus to CR1, presence/absence of CR1 and CR2, are shown for each species. Multiple sequence alignments for CR1 and CR2 are at bottom. The position of M144V in CR2 is indicated by the red asterisk. Identical and similar amino acids are highlighted in dark and light blue, respectively. FIG. 2(b) shows the results of a subcellular localization assay. Transiently expressed myc-tagged C7orf11 in human cultured cells (HEK293 and VA13) were examined by immunostaining using anti-myc antibody and a secondary antibody conjugated with FITC. BF (bright field), DAPI, FITC, and the merged image of DAPI and FITC signals are shown for each cell line. FIG. 1(c) shows the results of in situ RNA hybridization for C7orf11 on human developmental skin tissue. [35S]UTP-labeled riboprobes (sense and antisense) were hybridized to the sections. Radioactive signals were detected by autoradiography with 38 days exposure. Slides were counterstained with hematoxylin-eosin and photographed under dark-field (DF) and bright-field (BF). The signal detected in epidermis and hair follicles is specific to the antisense probe.

### DETAILED DESCRIPTION OF THE INVENTION

The invention identifies C7orf11 as the disease gene responsible for non-photosensitive TTD in a subset of non-photosensitive TTD afflicted patients. In aspects, this subset may comprise about 10% of TTD. C7orf11 is the first gene identified to be mutated in non-photosensitive TTD patients. Mutations in C7orf11 lead to the development of non-photosensitive TTD. Further, absence of the gene leads to the development of non-photosensitive TTD. With the knowledge that mutations in the C7orf11 sequence are causal of non-photosensitive TTD in a sub-group of patients, the genomic, cDNA and protein sequences thereof can be used in a variety of methods for the screening of the disease, for diagnosis of the disease, for developing therapies for treatment of disease, for developing pharmacological therapies of the disease and for the development of animal models of the disease. The knowledge of mutations causative of non-photosensitive TTD in the C7orf11 nucleic acid sequence is particularly beneficial for carrier detection, DNA diagnosis and family counseling. Men and women (of child-bearing age) may be screened for carrier status. Carrier detection using probes specific to a mutation or by using RFLP analysis are useful. Other carriers may be detected using probes specific for various haplotype groups. Prenatal diagnosis is useful to assess whether a fetus will be born with non-photosensitive TTD or be a carrier thereof. Prenatal diagnosis is also useful to determine whether a child will be born with a symptom or after birth develop a symptom selected from the group consisting of brittle hair, nail dystrophy, mental retardation, growth retardation, ichthyosis, decreased fertility, infertility and combinations thereof. The invention encompasses the screening and diagnosis of any human or fetus that may have or be predisposed to have a C7orf11 gene mutation including but not limited to suspected TTD subjects, non-photosensitive TTD subjects and ABHS subjects.

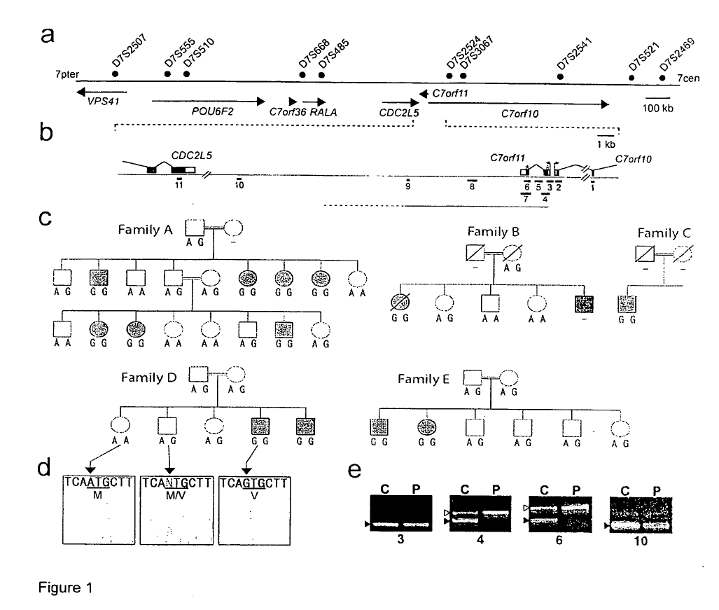

The C7orf11 gene was isolated from a 2 Mb locus on human chromosome 7p14. The genomic sequence for the C7orf11 gene is shown in Table 1 as Sequence ID No. 1. The cDNA sequence is shown in Table 2 represented as Sequence ID No. 2. It is seen from Table 2 and FIG. 1 that the C7orf11 consists of two exons spanning a 2 kb region. The amino acid sequence of the C7orf11 protein is shown in Table 3 and represented by Sequence ID No. 3. FIG. 1(b) shows the position of the M144V mutation and the 2 bp deletion in the Amish kindred.

In aspects of the invention, the mutations comprise a homozygous A>G DNA sequence alteration in the C7orf11 gene sequence causing the amino acid substitution M144V. Mutations also comprise deletions in one or both of the two exons of the C7orf11 gene. Such deletions may lead to the expression of a truncated protein or deletion of a substantial part or an entire exon. One such deletion mutation identified is a 2 bp homozygous deletion in exon 1 (corresponding to nucleotides 187 and 188 of Sequence ID No. 1). This is predicted to create a 57 amino acid truncated protein. Other identified deletions include a deletion of the entire exon 1 or part of exon 1 and the entire of exon 2 of Sequence ID No. 2 leading to a patient being genetically null for the C7orf11 gene.

The absence of cutaneous photosensitivity in the patients with C7orf11 mutations along with the protein's nuclear localization, it is possible that C7orf11 might be involved in transcription but not DNA repair. Moreover, the brittle hair observed in TTD patients is thought to result from the reduced expression of high sulfur proteins (intermediate keratin filaments and matrix proteins) in the late stage of keratinocyte differentiation. Genes in these pathways and/or proteins associated with C7orf11 would be the primary candidates to be involved in other non-photosensitive TTD cases.

To identify the gene causal for non-photosensitive TTD, the ABHS disease locus was searched. Homozygosity mapping was performed on a subset of affected members from a consanguineous Amish kindred7. This mapping identified a 2 Mb candidate locus on 7p14 (FIG. 1a and Seboun, E. et al. submitted). Then 7 genes were analyzed (FIG. 1b and (11)) by DNA sequencing (Table 5). A homozygous A>G variant in the C7orf11 gene was identified in the affected members in Family E (FIG. 1c) that causes an amino acid (aa) substitution (M144V). The Applicant has previously shown that C7orf11 encodes a 179 amino acid protein of unknown function^{11,12} and that it demonstrates variable expression in many tissues including fibroblasts and brain. By sequencing all available Amish kindred members, it was confirmed that the 13 affected cases were homozygous for the A>G variant, and 26 unaffected members were either heterozygous carriers (18/26) or homozygous for the normal allele (8/26) (FIGS. 1c and 1d). The 148 controls (296 chromosomes) were genotyped including 48 unrelated Amish and the co-segregation of the M144V variant was confirmed only in carrier or disease chromosomes.

The C7orf11 gene was then examined in twelve additional non-photosensitive TTD cases and deleterious homozygous deletions were found in two individuals. The fibroblasts derived from all but two Sabinas patients were tested for ultraviolet (UV) sensitivity using various NER parameters, including unscheduled DNA synthesis, recovery from transcription-inhibition, and overall clonogenic cell survival after UV exposure. In TTD siblings of Moroccan origin¹³, a 2 bp homozygous deletion in exon 1 was found (nt 187 and 188 of NM_{—}138701, Sequence ID No. 1) (data not shown), which is predicted to create a 57 amino acid truncated protein. In the second case, an Italian TTD patient with severe nervous system impairment¹⁴ (Pt. 6474), the attempts to amplify the coding regions of C7orf11 failed. By multiplex PCR using a primer pair that amplifies a 704 bp control fragment, it was determined that a part of exon 1 and the entire exon 2 of C7orf11 were homozygously deleted, while the flanking genes (CDC2L5 and C7orf10) were not (FIGS. 1b and 1e). Therefore, the patient is likely to be genetically null for C7orf11, which might explain the more severe neurological phenotype when compared to the ABHS patients. Mutations were not found in the two exons and 5′ upstream region of C7orf11 in the other ten non-photosensitive TTD cases including two Sabinas and one Pollitt patients, suggesting genetic heterogeneity exists in non-photosensitive TTD. Microsatellite analysis also excluded the 2 Mb C7orf11 locus from involvement in Sabinas syndrome.

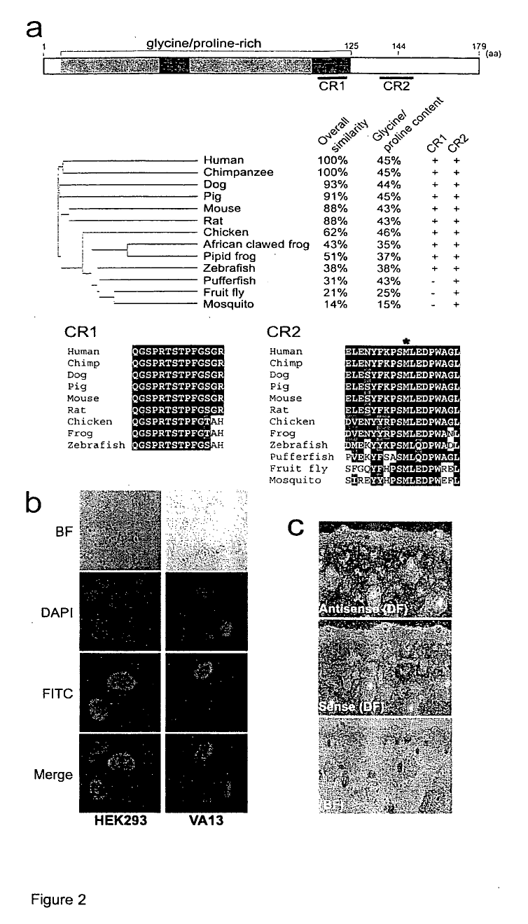

Proteins with predicted sequence similarity to human C7orf11 were identified from six mammalian species in addition to chicken, frog, fish, and insects, but not in lower eukaryotic species (C. elegans and yeast). The first two thirds of the human C7orf11 protein is remarkably glycine/proline-rich (45% in 125 aa), and this feature is more evident in higher eukaryotic species (FIG. 2a). There are two highly conserved regions: CR1 conserved from zebra fish to human, and CR2 conserved from mosquito to human (FIG. 2a). The mutant M144V found in ABHS patients is located in the three aa residues (SML) in CR2, conserved in all species. To examine the subcellular localization of the C7orf11, Myc-epitope tagged protein was transfected into cultured mammalian cells and found it predominantly in the nucleus (FIG. 2b). The pattern of the Myc-epitope mutated protein was indistinguishable from the wild type (data not shown). In situ RNA hybridization analysis was performed for C7orf11 in human early developmental skin tissue, and found it expressed in epidermis and hair follicles consistent with presentation of the phenotype (FIG. 2C). C7orf11 expression was not clearly detected in dermis by in situ hybridization, but it was found by RT-PCR in fibroblast cells.

Accordingly the invention provides various screening and diagnostic uses for the nucleotide sequence of C7orf11, both genomic and cDNA sequence as well as for antibodies which recognize both normal and mutant forms of the sequence. The invention also provides the manufacture of the protein for therapy as compositions, gene therapy methods, kits, and animal models for the study of the disease and to help elucidate pharmacological agents which can alleviate the symptoms of non-photosensitive TTD in patients as well as treat and prevent the disease.

Thus, in accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Nucleic Acid

The invention encompasses nucleic acid molecules, in aspects DNA molecules encoding a normal or mutant C7orf11 protein. Such nucleic acid molecules may comprise (A) an isolated nucleotide sequence as set forth in SEQ ID NO: 1 or SEQ ID NO: 2; (B) isolated nucleic acid molecules that comprise nucleic acid sequences that hybridize under high stringency conditions to the isolated DNA as set forth in SEQ ID NO: 1 or SEQ ID NO: 2; and nucleic acid sequences that hybridize to (1) or (2), above. The invention also encompasses nucleic acid molecules comprising a mutation in SEQ ID NO: 1 or SEQ ID NO: 2. that lead to the development of non-photosensitive TTD or symptoms of TTD as well as isolated nucleic molecules that hybridize under high stringency conditions to the isolated DNA mutant sequences. Such hybridization conditions may be highly stringent or less highly stringent, as is understood by one of skill in the art. In instances wherein the nucleic acid molecules are deoxyoligonucleotides (“oligos”), highly stringent conditions may refer, for example but not limited to, to washing in 6×SSC/0.05% sodium pyrophosphate at 37° C. (for 14-base oligos), 48° C. (for 17-base oligos), 55° C. (for 20-base oligos), and 60° C. (for 23-base oligos). Suitable ranges of such stringency conditions for nucleic acids of varying compositions are described in Krause and Aaronson (1991) Methods in Enzymology, 200:546-556 in addition to Maniatis et al., cited above.

The invention also encompasses nucleic acid sequences having sequence homology to the isolated DNA as set forth in SEQ ID NO: 1 or SEQ ID NO: 2; and in aspects, nucleic acid sequences sharing up to about 80%, up to about 90% and up to about 95% sequence homology to the sequences set forth in SEQ ID NO or SEQ ID NO. Furthermore, such nucleic acid sequence sharing sequence homology may contain one or more mutations as described herein that are causative of non-photosensitive trichothiodystrophy.

The invention encompasses various fragments of a C7orf11 nucleic acid for use in diagnosis of and/or prediction of the likelihood of carrying a mutant C7orf11 nucleic acid sequence or exhibiting symptoms of non-photosensitive TTD, such a fragment means any fragment that can be used as specific probe in hybridization or as a primer in PCR reactions. Fragments of the C7orf11 nucleic acid sequence (genomic or cDNA) can comprise about 9 nucleotides or more as is understood by those of skill in the art and can be used as probes or primers. A functional fragment of a nucleic acid for use in the treatment of non-photosensitive TTD means any fragment that can be transcribed and/or translated into a functional protein. A functional fragment of a C7orf11 gene product for the manufacture of a medicament to treat non-photosensitive TTD is any fragment of gene product that retains its normal function such that non-photosensitive TTD does not develop in a patient. A functional fragment of a C7orf11 gene product in the production of antibodies is an immunogenic fragment that comprises at least one epitope and can be used for the production of antibodies against the gene product. A functional fragment of a C7orf11 gene product in the isolation of an interacting compound is any fragment that can be used in an interaction screening assay, such as, but not limited to, a yeast two-hybrid assay, a phage display assay, co-immunoprecipitation, a DNase protection assay, an electrophoretic mobility shift assay, or mass spectrometric analyses.

The terms protein and polypeptide as used in this application are interchangeable. Polypeptide refers to a polymer of amino acids and does not refer to a specific length of the molecule. This term also includes post-translational modifications of the polypeptide, such as glycosylation, phosphorylation and acetylation. Compound as used here means any chemical or biological compound, including simple or complex organic or inorganic molecules, peptides, peptido-mimetics, proteins, antibodies, carbohydrates, nucleic acids or derivatives thereof. Interacting compound with a protein means any compound that can bind, covalently or not, with said protein in a specific way.

The invention also encompasses (a) vectors that contain any of the foregoing coding sequences (i.e., sense) and/or their complements (i.e., antisense); (b) expression vectors that contain any of the foregoing coding sequences operatively associated with a regulatory element that directs the expression of the coding sequences; and (c) genetically engineered host cells that contain any of the foregoing coding sequences operatively associated with a regulatory element that directs the expression of the coding sequences in the host cell. As used herein, regulatory elements include but are not limited to inducible and non-inducible promoters, enhancers, operators and other elements known to those skilled in the art that drive and regulate expression. As used herein a “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication, i.e., capable of replication under its own control.

As described above, the C7orf11 gene or mutant gene sequence (or fragments thereof) can be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pMal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast plasmid.

More specifically, a variety of host-expression vector systems may be utilized to express the differentially expressed gene coding sequences of the invention. Such host-expression systems represent vehicles by which the coding sequences of interest may be produced and subsequently purified, but also represent cells which may, when transformed or transfected with the appropriate nucleotide coding sequences, express the differentially expressed gene protein of the invention in situ. These include but are not limited to microorganisms such as bacteria (e.g., E. coli, B. subtilis) transformed with recombinant bacteriophage DNA, plasmid DNA or cosmid DNA expression vectors containing differentially expressed gene protein coding sequences; yeast (e.g. Saccharomyces, Pichia) transformed with recombinant yeast expression vectors containing the differentially expressed gene protein coding sequences; insect cell systems infected with recombinant virus expression vectors (e.g., baculovirus) containing the differentially expressed gene protein coding sequences; plant cell systems infected with recombinant virus expression vectors (e.g., cauliflower mosaic virus, CaMV; tobacco mosaic virus, TMV) or transformed with recombinant plasmid transformation vectors (e.g., Ti plasmid) containing differentially expressed gene protein coding sequences; or mammalian cell systems (e.g. COS, CHO, BHK, 293, 3T3) harboring recombinant expression constructs containing promoters derived from the genome of mammalian cells (e.g., metallothioneine promoter) or from mammalian viruses (e.g., the adenovirus late promoter; the vaccinia virus 7.5K promoter; Cytomegalovirus Early gene promoter).

In bacterial systems, a number of expression vectors may be advantageously selected depending upon the use intended for the differentially expressed gene protein being expressed. For example, when a large quantity of such a protein is to be produced, for the generation of antibodies or to screen peptide libraries, for example, vectors which direct the expression of high levels of fusion protein products that are readily purified may be desirable. Such vectors include, but are not limited, to the E. coli expression vector pUR278 (Ruther et al., 1983, EMBO J. 2:1791), in which the differentially expressed gene protein coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. PGEX vectors may also be used to express foreign polypeptides as fusion proteins with glutathione S-transferase (GST). In general, such fusion proteins are soluble and can easily be purified from lysed cells by adsorption to glutathione-asephagarose beads followed by elution in the presence of free glutathione. The PGEX vectors are designed to include thrombin or factor Xa protease cleavage sites so that the cloned target gene protein can be released from the GST moiety by using these endopeptidases.

Promoter regions can be selected from any desired gene using vectors that contain a reporter transcription unit lacking a promoter region, such as a chloramphenicol acetyl transferase (“cat”) or luciferase transcription unit, downstream of restriction site or sites for introducing a candidate promoter fragment; i.e., a fragment that may contain a promoter. As is well known, introduction into the vector of a promoter-containing fragment at the restriction site upstream of the cat or luciferase gene engenders production of CAT or luciferase activity, which can be detected by standard CAT assays or luminometry. Vectors suitable to this end are well known and readily available. Three such vectors are pKK232-8, -pCM7 and pGL3 (Promega, E1751, Genebank Assn. No. u47295). Thus, promoters for expression of polynucleotides of the present invention include not only well known and readily available promoters, but also promoters that readily may be obtained by the foregoing technique, using a reporter gene assay.

Among known bacterial promoters suitable for expression of polynucleotides and polypeptides in accordance with the present invention are the E. coli laci and lacZ promoters, the T3 and T7 promoters, the T5 tac promoter, the lambda PR, PL promoters and the trp promoter. Among known eukaryotic promoters suitable in this regard are the CMV immediate early promoter, the HSV thymidine kinase promoter, the early and late SV40 promoters, the promoters of retroviral LTRS, such as those of the Rous sarcoma virus (“RSV”), and metallothionein promoters, such as the mouse metallothionein-I promoter.

In an insect system, Autographa californica nuclear polyhedrosis virus (AcNPV) is one of several insect systems that can be used as a vector to express foreign genes. The virus grows in Spodoptera frugiperda cells. The differentially expressed gene coding sequence may be cloned individually into non-essential regions (for example the polyhedrin gene) of the virus and placed under control of an AcNPV promoter (for example the polyhedrin promoter). Successful insertion of differentially expressed gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (E.g., see Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the differentially expressed gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing differentially expressed gene protein in infected hosts. (e.g., See Logan & Shenk, 1984, Proc. Natl. Acad. Sci. USA 81:3655-3659). Specific initiation signals may also be required for efficient translation of inserted differentially expressed gene coding sequences. These signals include the ATG initiation codon and adjacent sequences. In cases where an entire differentially expressed gene, including its own initiation codon and adjacent sequences, is inserted into the appropriate expression vector, no additional translational control signals may be needed. However, in cases where only a portion of the differentially expressed gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals (Kozack sequence) and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544).

The C7orf11 cDNA may be used for the purposes of making other mutations to identify further mutation(s) responsible for the loss or alteration of function of the mutant gene product. Alternatively, a genomic or cDNA library can be constructed and screened using DNA or RNA, from a tissue known to or suspected of expressing the C7orf11 gene in an individual suspected of to carry the mutant allele. The normal gene or any suitable fragment thereof may then be labeled and used as a probe to identify the corresponding mutant allele in the library. The clone containing this gene may then be purified through methods routinely practiced in the art, and subjected to sequence analysis as described above.

Additionally, an expression library can be constructed utilizing DNA isolated from or cDNA synthesized from a tissue known to or suspected of expressing the gene of interest in an individual suspected of to carry a mutant allele. In this manner, gene products made by the putatively mutant tissue may be expressed and screened using standard antibody screening techniques in conjunction with antibodies raised against the normal gene product, as described, below. (For screening techniques, see, for example, Harlow, E. and Lane, eds., 1988, “Antibodies: A Laboratory Manual”, Cold Spring Harbor Press, Cold Spring Harbor.) In cases where the mutation results in an expressed gene product with altered function (e.g., as a result of a missense mutation), a polyclonal set of antibodies are likely to cross-react with the mutant gene product. Library clones detected via their reaction with such labeled antibodies can be purified and subjected to sequence analysis as described above.

In addition, expressed gene products may include proteins that represent functionally equivalent gene products. Such an equivalent gene product may contain deletions, additions or substitutions of amino acid residues within the amino acid sequence encoded by the differentially expressed gene sequences described, above, but which result in a silent change, thus producing a functionally equivalent differentially expressed gene product (polymorphisms). Amino acid substitutions may be made on the basis of similarity in polarity, charge, solubility, hydrophobicity, hydrophilicity, and/or the amphipathic nature of the residues involved and on comparison with amino-acids sequence from other species. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Functionally equivalent,” as utilized herein, may refer to a protein or polypeptide capable of exhibiting a substantially similar in vivo or in vitro activity as the endogenous differentially expressed gene products encoded by the differentially expressed gene sequences described above. “Functionally equivalent” may also refer to proteins or polypeptides capable of interacting with other cellular or extracellular molecules in a manner similar to the way in which the corresponding portion of the endogenous differentially expressed gene product would. For example, a “functionally equivalent” peptide would be able, in an immunoassay, to diminish the binding of an antibody to the corresponding peptide (i.e., the peptidic amino acid sequence of which was modified to achieve the “functionally equivalent” peptide) of the endogenous protein, or to the endogenous protein itself, where the antibody was raised against the corresponding peptide of the endogenous protein. An equimolar concentration of the functionally equivalent peptide will diminish the aforesaid binding of the corresponding peptide by at least about 5%, preferably between about 5% and 10%, more preferably between about 10% and 25%, even more preferably between about 25% and 50%, and most preferably between about 40% and 50%.

Methods which are well known to those skilled in the art can be used to construct expression vectors containing expressed gene protein coding sequences and appropriate transcriptional/translational control signals. These methods include, for example, in vitro recombinant DNA techniques, synthetic techniques and in vivo recombination/genetic recombination. See, for example, the techniques described in Sambrook et al., 1989, supra, and Ausubel et al., 1989, supra. Alternatively, RNA or cDNA capable of encoding expressed gene protein sequences may be chemically synthesized using, for example, synthesizers. See, for example, the techniques described in “Oligonucleotide Synthesis”, 1984, Gait, M. J. ed., IRL Press, Oxford, which is incorporated by reference herein in its entirety.

Selection of appropriate vectors and promoters for expression in a host cell is a well known procedure and the requisite techniques for expression vector construction, introduction of the vector into the host and expression in the host per se are routine skills in the art. Generally, recombinant expression vectors will include origins of replication, a promoter derived from a highly-expressed gene to direct transcription of a downstream structural sequence, and a selectable marker to permit isolation of vector containing cells after exposure to the vector. In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, etc.

For long-term, high-yield production of recombinant proteins, stable expression is preferred. For example, cell lines which stably express the differentially expressed protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed protein. Such engineered cell lines may be particularly useful in screening and evaluation of compounds that affect the endogenous activity of the differentially expressed protein. These stable cell lines might by used as a way of cellular therapy directly or after encapsulation.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk.sup.-, hgprt.sup.- or apr.sup.-cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

An alternative fusion protein system allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni.sup.2+ nitriloacetic acid-agarose columns and histidine-tagged proteins are selectively eluted with imidazole-containing buffers. When used as a component in assay systems such as those described below, the differentially expressed protein may be labeled, either directly or indirectly, to facilitate detection of a complex formed between the differentially expressed protein and a test substance. Any of a variety of suitable labeling systems may be used including but not limited to radioisotopes such as .sup.125I; enzyme labeling systems that generate a detectable calorimetric signal or light when exposed to substrate; and fluorescent labels.

Where recombinant DNA technology is used to produce the differentially expressed protein for such assay systems, it may be advantageous to engineer fusion proteins that can facilitate labeling, immobilization and/or detection. Indirect labeling involves the use of a protein, such as a labeled antibody, which specifically binds to either a differentially expressed gene product. Such antibodies include but are not limited to polyclonal, monoclonal, chimeric, single chain, Fab fragments and fragments produced by a Fab expression library.

The C7orf11 peptides, normal or mutant and fragments of both thereof, of the invention may also be obtained by chemical synthesis using automated instruments. The peptides of the invention may be prepared by chemical synthesis using techniques well known in the chemistry of proteins such as solid phase synthesis (Merrifield, J. Am. Chem. Assoc. 85:2149-2154 (1964)) or synthesis in homogenous solution (Houbenweyl, Methods of Organic Chemistry (1987), (Ed. E. Wansch) Vol. 15, pts. I and II, Thieme, Stuttgart).

A functional fragment of the C7orf11 gene product, means any proteinous molecule that retains its normal activity. A variant of the C7orf11 gene product according to the invention is a gene product with at least 60% identity, preferably 70% identity, more preferably 80% identity, most preferably 90% identity to said C7orf11 gene product, as measured by a BLASTP or TBLASTN search (Altschul et al., 1997), and retaining its normal activity.

Protein Therapy

Therapies may be designed to circumvent or overcome a C7orf11 gene defect or inadequate C7orf11 gene expression, and thus moderate and possibly prevent development of non-photosensitive TTD or one or more symptoms of the disease. The C7orf11 gene has been found to be mutated or essentially not expressed in non-photosensitive TTD. In considering various therapies, however, it is understood that such therapies may be targeted at a variety of cells/tissues in which C7orf11 is demonstrated to be expressed or have a function.

Treatment or prevention of non-photosensitive TTD or any symptoms thereof can be accomplished by replacing a mutant C7orf11 protein with normal protein, by modulating the function of a mutant protein, or by delivering normal C7orf11 protein to the appropriate cells. It may also be possible to modify the pathophysiologic pathway (signal transduction pathway) in which the protein participates in order to correct the physiological defect.

To replace a mutant protein with normal protein, or to add protein to cells which no longer express C7orf11, or express inadequate amounts of C7orf11 it is necessary to obtain large amounts of pure C7orf11 protein from cultured cell systems which can express the protein. Delivery of the protein to the affected cells and tissues can then be accomplished using appropriate packaging or administration systems. Alternatively, small molecule analogs may be used and administered to act as C7orf11 agonists or antagonists and in this manner produce a desired physiological effect. In order to screen for analogues, one can design functional screens based on the sequence of C7orf11. One can also fuse C7orf11 to heterologous DNA binding proteins to design screens for agonists. Yeast screens can be used for small molecules that may interact by promoting or disrupting C7orf11 binding with other proteins.

Based on the biochemical analyses of C7orf11 protein structure-function, one can design drugs to mimic the effects of C7orf11 on target proteins. Recombinant C7orf11 expressed as a fusion protein can be utilized to identify small peptides that bind to C7orf11 such as by using a phage display approach. An alternate but related approach uses the yeast two hybrid system to identify further binding partners for C7orf11. C7orf11 or fragments thereof are expressed in yeast as a fusion to a DNA binding domain. This fusion protein is capable of binding to target promoter elements in genes that have been engineered into the yeast. These promoters drive expression of specific reporter genes (typically the auxotrophic marker HIS3 and the enzyme β-galactosidase). A library of cDNAs can then be constructed from any tissue or cell line and fused to a transcriptional activation domain. Transcription of HIS3 and β-galactosidase depends on association of the C7orf11 fusion protein (which contains the DNA binding domain) and the target protein (which carries the activation domain). Yeast survival on specific growth media lacking histidine requires this interaction. This approach allows for the identification of specific proteins that interact with C7orf11. The approach has also been adapted to identify small peptides. C7orf11, or its fragments, are fused with the DNA binding domain and are screened with a library of random peptides or peptides which are constrained at specific positions linked to a transcriptional activation domain. Interaction is detected by growth of the interacting peptides on media lacking histidine and by detection of β-galactosidase activity using standard techniques.

The identification of proteins or small peptides that interact with normal or mutant forms of C7orf11 can provide the basis for the design of small peptide antagonists or agonists of C7orf11 function. Further, the structure of these peptides determined by standard techniques such as protein NMR or X-ray crystallography can provide the structural basis for the design of small molecule drugs.

Gene Therapy

In another embodiment, nucleic acids comprising a sequence encoding C7orf11 protein or functional derivative thereof, are administered to promote normal function, by way of gene therapy. Gene therapy refers to therapy performed by the administration of a nucleic acid to a subject. In this embodiment of the invention, the nucleic acid produces its encoded protein that mediates a therapeutic effect by promoting normal function.

Any of the methods for gene therapy available in the art can be used according to the present invention. Exemplary methods are described below. In a preferred aspect, the therapeutic comprises a C7orf11 nucleic acid that is part of an expression vector that expresses a normal C7orf11 protein or fragment or chimeric protein thereof in a suitable host. In particular, such a nucleic acid has a promoter operably linked to the C7orf11 coding region, said promoter being inducible or constitutive, and, optionally, tissue-specific. In another particular embodiment, a nucleic acid molecule is used in which the C7orf11 coding sequences and any other desired sequences are flanked by regions that promote homologous recombination at a desired site in the genome, thus providing for intrachromosomal expression of the C7orf11 nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy. In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (adenovirus, adeno-associated virus and lentivirus) (see, e.g., U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., U.S. Pat. Nos. 5,166,320; 5,728,399; 5,874,297; and 6,030,954, all of which are incorporated by reference herein in their entirety) (which can be used to target cell types specifically expressing the receptors), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188; and WO 93/20221). Alternatively, the nucleic acid can be introduced intracellularly and incorporated within host cell DNA for expression, by homologous recombination (see, e.g., U.S. Pat. Nos. 5,413,923; 5,416,260; and 5,574,205; and Zijlstra et al., 1989, Nature 342:435-438).

In a specific embodiment, a viral vector that contains the C7orf11 nucleic acid is used. For example, a retroviral vector can be used (see, e.g., U.S. Pat. Nos. 5,219,740; 5,604,090; and 5,834,182). These retroviral vectors have been modified to delete retroviral sequences that are not necessary for packaging of the viral genome and integration into host cell DNA. The C7orf11 nucleic acid to be used in gene therapy is cloned into the vector, which facilitates delivery of the gene into a patient.

Adenoviruses are other viral vectors that can be used in gene therapy. Adenoviruses have the advantage of being capable of infecting non-dividing cells. Methods for conducting adenovirus-based gene therapy are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; 6,001,557; and 6,033,8843, all of which are incorporated by reference herein in their entirety. Adeno-associated virus (AAV) has also been proposed for use in gene therapy. Adeno-associated viruses are especially attractive vehicles for delivering genes to the retina. Methods for producing and utilizing AAV are described, e.g., in U.S. Pat. Nos. 5,173,414; 5,252,479; 5,552,311; 5,658,785; 5,763,416; 5,773,289; 5,843,742; 5,869,040; 5,942,496; and 5,948,675, all of which are incorporated by reference herein in their entirety.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient directly or after encapsulation.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. The resulting recombinant cells can be delivered to a patient by various methods known in the art.

Cells into which a nucleic acid can be introduced for purposes of gene therapy encompass various cell types such as but not limited to fibroblasts, brain cells, epidermal cells, keratinocytes and other cell that may express C7orf11 gene product or be benefited from receiving a C7orf11 gene product. In a preferred embodiment, the cell used for gene therapy is autologous to the patient. In an embodiment in which recombinant cells are used in gene therapy, a C7orf11 nucleic acid is introduced into the cells such that it is expressible by the cells or their progeny, and the recombinant cells are then administered in vivo for therapeutic effect. In a further embodiment, stem or progenitor cells may be used. Any stem- and/or progenitor cells which can be isolated and maintained in vitro can potentially be used in accordance with this embodiment of the present invention. For example, epithelial stem cells (ESCs) or keratinocytes can be obtained from tissues such as the skin and the lining of the gut by known procedures (Rheinwald, 1980, Meth. Cell Bio. 21A:229). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Pittelkow and Scott, 1986, Mayo Clinic Proc. 61:771). If the ESCs are provided by a donor, a method for suppression of host versus graft reactivity (e.g., irradiation, drug or antibody administration to promote moderate immunosuppression) can also be used. Retinal stem cells (Tropepe et al., 2000, Science, 287: 2032).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Antibodies

Described herein are methods for the production of antibodies capable of specifically recognizing one or more differentially expressed gene epitopes whether normal or mutant. Such antibodies may include, but are not limited to polyclonal antibodies, monoclonal antibodies (mAbs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Such antibodies may be used, for example, in the detection of a fingerprint, target, gene in a biological sample, or, alternatively, as a method for the inhibition of abnormal target gene activity. Thus, such antibodies may be utilized as part of disease treatment methods, and/or may be used as part of diagnostic techniques whereby patients may be tested for normal or mutant forms of C7orf11 Ophthalmology 87,313-319)].

For the production of antibodies that recognize the normal or mutant C7orf11 polypeptide, or fragments thereof, various host animals may be immunized by injection with an expressed protein, or a portion thereof or by DNA immunization. Such host animals may include but are not limited to rabbits, mice, and rats, to name but a few. The purified C7orf11 protein (normal or mutant or antigenic fragment thereof) is purified, coupled to a carrier protein and mixed with Freund's adjuvant (to help stimulate the antigenic response by the animal) and injected into rabbits or other appropriate laboratory animals. Following booster injections at weekly intervals, the rabbits or other laboratory animals are then bled and the sera isolated. The sera can be used directly or purified prior to use by various methods including affinity chromatography employing Protein A-Sepharose, Antigen Sepharose or Anti-mouse-Ig-Sepharose, to give polyclonal antibodies. Alternatively, synthetic peptides can be made corresponding to the antigenic portions of the protein and used to inoculate the animals. The most common practice is to choose a 10 to 15 amino acid residue peptide corresponding to the carboxyl or amino terminal sequence of a protein antigen, and to chemically cross-link it to a carrier molecule such as keyhole limpet hemocyanin or BSA. However, if an internal sequence peptide is desired, selection of the peptide is based on the use of algorithms that predict potential antigenic sites. These predictive methods are, in turn, based on predictions of hydrophilicity (Kyte and Doolittle 1982, Hopp and Woods 1983) or secondary structure (Chou and Fasman 1978). The objective is to choose a region of the protein that is either surface exposed, such as a hydrophilic region, or is conformationally flexible relative to the rest of the structure, such as a loop region or a region predicted to form a β-turn. The selection process is also limited by constraints imposed by the chemistry of the coupling procedures used to attach peptide to carrier protein. Carboxyl-terminal peptides are frequently chosen because these are often more mobile than the rest of the molecule and the peptide can be coupled to a carrier in a straightforward manner using glutaraldehyde. The amino-terminal peptide has the disadvantage that it may be modified post-translationally by acetylation or by the removal of a leader sequence. A comparison of the protein amino acid sequence between species can yield important information. Those regions with sequence differences between species are likely to be immunogenic. Synthetic peptides can also be synthesized as immunogens as long as they mimic the native antigen as closely as possible.

Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with -differentially expressed gene product supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgQ, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce differentially expressed gene-single chain antibodies. Single chain antibodies are formed by linking the heavy and light chain fragments of the Fv region via an amino acid bridge, resulting in a single chain polypeptide.

Furthermore, techniques useful for the production of “humanized antibodies” can be adapted to produce antibodies to the polypeptides, fragments, derivatives, and functional equivalents disclosed herein. Such techniques are disclosed in U.S. Pat. Nos. 5,932,448; 5,693,762; 5,693,761; 5,585,089; 5,530,101; 5,910,771; 5,569,825; 5,625,126; 5,633,425; 5,789,650; 5,545,580; 5,661,016; and 5,770,429, the disclosures of all of which are incorporated by reference herein in their entirety.

Antibody fragments which recognize specific epitopes may be generated by known techniques. For example, such fragments include but are not limited to: the F(ab′).sub.2 fragments which can be produced by pepsin digestion of the antibody molecule and the Fab fragments which can be generated by reducing the disulfide bridges of the F(ab).sub.2 fragments. Alternatively, Fab expression libraries may be constructed (Huse et al., 1989, Science, 246:1275-1281) to allow rapid and easy identification of monoclonal Fab fragments with the desired specificity.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the unlabeled and the labeled antibodies be a C7orf11 antibody.

Commonly used reporter molecules in this type of assay are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex of antibody-RdCVF1- or RdCVF2-labeled antibody. The substrate reacts with the enzyme linked to the second antibody, giving a qualitative visual signal, which may be further quantitated, usually spectrophotometrically, to give an evaluation of the amount of Rdcvf1 or Rdcvf2 which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

It is understood by those of skill in the art that antibodies that detect both normal and various mutant forms of the C7orf11 gene product are useful in the identification of carriers of the disease as well as for diagnostic purposes.

Diagnostic

The invention also relates to the use of polynucleotides of the present invention as diagnostic reagents. Detection of a mutated form of the gene encoding a polypeptide which is associated with a dysfunction will provide a diagnostic tool that can add to, or define, a diagnosis of non-photosensitive TTD disease; or susceptibility to the disease, which results from under-expression, over-expression or altered spatial or temporal expression of the gene; no expression of the gene; or susceptibility of developing any of the symptoms of the disease such as but not limited to mental retardation, decreased fertility and infertility as described herein. Such susceptibility is more likely to be diagnosed in fetal subjects or younger subjects which may not yet have developed certain phenotypic symptoms of the disease. Individuals carrying mutations in the gene may be detected at the DNA level by a variety of techniques. In aspects, the invention comprises a method for determining whether a subject displaying non-photosensitive TTD-associated symptoms suffers from non-photosensitive TTD, or an asymptomatic subject is a carrier of non-photosensitive TTD, the method comprising: obtaining a biological sample from the subject; and conducting an assay on the sample to determine the presence or absence of at least one mutation in the 2 Mb 7p14 region of chromosome 7, the presence of at least one mutation being indicative that the subject displaying symptoms suffers from non-photosensitive TTD or that the asymptomatic subject is a carrier of non-photosensitive TTD.

In an embodiment, nucleic acids for diagnosis or carrier detection may be obtained from a subjects cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents (SSCP), or by direct DNA sequencing (e.g., Myers et al., Science (1985) 230:1242). Sequence changes at specific locations may also be revealed by nuclease protection assays, such as RNase and SI protection or the chemical cleavage method (see Cotton et al., Proc Natl Acad Sci USA (1985) 85: 4397-4401). In another embodiment, an array of oligonucleotides probes comprising the C7orf11 nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al., Science, Vol 274, pp 610-613 (1996)).

The diagnostic assays offer a process for diagnosing or determining a susceptibility to disease through detection of mutation in the C7orf11 gene by the methods described. In addition, such diseases may be diagnosed by methods comprising determining from a sample derived from a subject an abnormal C7orf11 expression. Expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays.

Thus in another aspect, the present invention relates to a diagnostic kit which comprises: (a) a polynucleotide of the present invention, preferably the nucleotide sequence encoding a polypeptide SEQ ID NO: 3 or a fragment thereof, or a mutant form of SEQ ID NO: 3, (b) a nucleotide sequence complementary to that of (a); (c) a normal or mutant polypeptide of the present invention, preferably the polypeptide of or a fragment thereof; or (d) an antibody to a polypeptide of the present invention, preferably to a polypeptide of SEQ ID NO or a mutant form thereof. Such a kit will be of use in diagnosing a disease or susceptibility to a disease, particularly non-photosensitive TTD.

The invention also relates to the use of antibodies which recognize both normal or mutant C7orf11 polypeptides of the present invention as diagnostic reagents. Detection of a mutated form of the C7orf11 polypeptide which is associated with a dysfunction will provide a diagnostic tool that can add to, or define, a diagnosis of non-photosensitive TTD disease; or susceptibility to the disease; or susceptibility of developing any of the symptoms of the disease such as but not limited to mental retardation, decreased fertility and infertility as described herein. Such susceptibility is more likely to be diagnosed in fetal subjects or younger subjects which may not yet have developed certain phenotypic symptoms of the disease. According to this aspect of the invention, is a kit for assaying for the presence of a mutant C7orf11 gene by immunoassay techniques comprises: (a) an antibody which specifically binds to a mutant gene product of the C7orf11 gene; (b) reagent means for detecting the binding of the antibody to the gene product; and (c) the antibody and reagent means each being present in amounts effective to perform the immunoassay.

Pharmaceutical Compositions

An additional embodiment of the invention relates to the development and administration of a pharmaceutical composition, in conjunction with a pharmaceutically acceptable carrier, for the treatment of any symptoms of non-photosensitive TTD. Such pharmaceutical compositions may comprise normal C7orf11 protein mimetics or agonists and fragments thereof. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions encompassed by the invention may be administered by any number of routes. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Pharmaceutical preparations which can be used orally include capsules made of gelatin, as well as soft, sealed capsules made of gelatin and a coating, such as glycerol or sorbitol. Such capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated m aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration of C7orf11 protein, such labeling would include amount, frequency, and method of administration. Pharmaceutical compositions suitable for use in the invention include compositions wherein the active ingredients are contained in an effective amount to achieve the intended purpose. The determination of an effective dose is well within the capability of those skilled in the art. For any compound, the therapeutically effective dose can be estimated initially either in cell culture assays, or in animal models, usually mice, rabbits, dogs, or pigs. The animal model may also be used to determine the appropriate concentration range and route of administration. Such information can then be used to determine useful doses and routes for administration in humans.

A therapeutically effective dose refers to that amount of active ingredient, for example C7orf11 protein or fragments thereof, antibodies to C7orf11, agonists, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

Transgenic Animal Models

The present invention also embodies transgenic animals that are developed to express mutant forms of the C7orf11 nucleic acid sequences as disclosed herein. Such animal models are useful to study the pathophysiology of non-photosensitive TTD and also to use for screening various nucleic acid based, antibody based, protein based and pharmacologically based treatments for non-photosensitive TTD. A transgenic animal according to the invention is an animal having cells that contain a transgene which was introduced into the animal or an ancestor of the animal at a prenatal (embryonic) stage. Transgenic animals can be selected from farm animals (such as pigs, goats, sheep, cows, horses, rabbits, and the like), rodents (such as rats, guinea pigs, mice, and the like), non-human primates (such as baboon, monkeys, chimpanzees, and the like), and domestic animals (such as dogs, cats, and the like). A transgenic animal can be created, for example, by introducing the C7orf11 gene into the male pronucleus of a fertilized oocyte by, e.g., microinjection, and allowing the oocyte to develop in a pseudopregnant female foster animal. The gene sequence may include appropriate promoter sequences, as well as intronic sequences and polyadenylation signal sequences. Methods for producing transgenic animals are disclosed in, e.g., U.S. Pat. Nos. 4,736,866 and 4,870,009 and Hogan et al., A Laboratory Manual, Cold Spring Harbor Laboratory, 1986. A transgenic founder animal can be used to breed additional animals carrying the transgene. A transgenic animal carrying one transgene can also be bred to another transgenic animal carrying a second transgene to create a “double transgenic” animal carrying two transgenes. Alternatively, two transgenes can be co-microinjected to produce a double transgenic animal. Animals carrying more than two transgenes are also possible. Furthermore, heterozygous transgenic animals, i.e., animals carrying one copy of a transgene, can be bred to a second animal heterozygous for the same transgene to produce homozygous animals carrying two copies of the transgene.

Several techniques known in the art can be used to introduce nucleic acid into animals to produce the founder lines of transgenic animals. Such techniques include, but are not limited to, pronuclear microinjection (U.S. Pat. No. 4,873,191); retrovirus mediated gene transfer into germ lines (Van der Putten et al., Proc. Nail. Acad. Sci., USA, 82:6148-6152 (1985)); gene transfection into embryonic stem cells (Gossler A et al., Proc Natl Acad Sci USA 83:9065-9069 (1986)); gene targeting into embryonic stem cells (Thompson et al., Cell, 56:313-321 (1989)); nuclear transfer of somatic nuclei (Schnieke A E et al., Science 278:2130-2133 (1997)); and electroporation of embryos.

For a review of techniques that can be used to generate and assess transgenic animals, skilled artisans can consult Gordon (Intl. Rev. Cytol., 115:171-229 (1989)), and may obtain additional guidance from, for example: Hogan et al, Manipulating the Mouse Embryo (Cold Spring Harbor Press, Cold Spring Harbor, N.Y. 1986); Krimpenfort et al., Bio/Technology, 9:844-847 (1991); Palmiter et al., Cell, 41:343-345 (1985); Kraemer et al., Genetic Manipulation of the Early Mammalian Embryo (Cold Spring Harbor Press, Cold Spring Harbor, N.Y. 1985); Hammer et al., Nature, 315:680-683 (1985); Purscel et al., Science, 244:1281-1288 (1986); Wagner et al., U.S. Pat. No. 5,175,385; and Krimpenfort et al., U.S. Pat. No. 5,175,384.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

Mutation Screening and Genotyping by SNaPshot Assay

To screen the two exons and 5′ upstream region of the C7orf11 gene, three sets of primer pairs were used (sequences and product size are listed):

| | |
|---|---|
| C7orf11-5upF/ex1R1 | |
| (5′-GTCTCAGATGGCATCGGTC-3′, | (SEQ ID No. 4) |
| 5′-GTTCTCCCACCGGAACTGTA-3′, | (SEQ ID No. 5) |
| | and 413 bp), |
| C7orf11ex1-F2/R3 | |
| (5′-GAACTGATGTGCCGTAGGGT-3′, | (SEQ ID No. 6) |
| 5′-AAGTAAGAGCTCGGCAAACG-3′, | (SEQ ID No. 7) |
| | and 510 bp), |
| and | |
| C7orf11ex2-F/R2 | |
| (5′-CAATGTGATTCCCGCTAACC-3′, | (SEQ ID No. 8) |
| 5′-TCATACCACAAAACCACAATAGC-3′, | (SEQ ID No. 9) |
| | and 627 bp). |

PCR was performed on 50 ng of genomic DNA in 25 μl scale reaction [20 mM Tris-HCl (pH 8.4), 50 mM KCl, 1.5 mM MgCl2, 0.2 mM dNTP, 100 ng each primer, and 1 Unit Taq polymerase (Invitrogen)]. The cycling conditions were: initial denaturation at 94° C. for 3 min, followed by 35 cycles of denaturation at 94° C. for 30 seconds, annealing at 58° C. for 30 seconds, and extension at 72° C. for 60 seconds. PCR products were purified using microCLEAN (Microsone Ltd), and used the purified DNA as sequencing template. We performed sequencing reactions using Big Dye terminator kit and obtained sequences using an ABI-3730 (Applied Biosystems).

The sequence variant (M144V) identified in the ABHS patients was examined in a collection of 148 (296 chromosomes) control DNA samples, consisting of 100 individuals from CEPH families and Caucasian 200 Panel, and 48 Amish individuals. Amplified and purified PCR product was subjected to the SNaPshot assay (Applied Biosystems), a single nucleotide primer extension method for the genotyping of SNPs. For SNaPshot primer extension reaction, 0.01 to 0.40 pmol of PCR product was used in a total volume of 10 μl reaction mixture containing 0.5 pmol primer (5′-GACTTTGGAAAATTATTTCAAGCCTTCA-3′) (SEQ ID No. 10) and 1.25 μl SNaPshot Ready Reaction Mix (Applied Biosystems). The cycling conditions for the reaction were 25 cycles at 95° C. for 10 seconds, 50° C. for 5 seconds and 60° C. for 30 seconds. One unit of shrimp alkali phosphatase was added, and incubated at 37° C. for 1 hr, followed by 75° C. for 15 min. The reactions were resolved on an ABI 310 Genetic Analyzer, and analyzed using GeneScan Analysis Software Version 2.1.

Deletion Analysis on the TTD Patient with Severe Nervous System Impairment (Pt.6474)

The homozygous deletion at the C7orf11 locus detected in the TTD patient (Pt.6474) was analyzed using the following eleven primer sets (the primer sequences and expected product size are listed in parentheses):

| | |
|---|---|
| #1_C7orf10ex2-F/R | |
| (5′-TGTGGACTCCCTTGCTAAGAAT-3′, | (SEQ ID No. 11) |
| 5′-GAAAAACCCAATCACCAAAATG-3′ | (SEQ ID No. 12) |
| | and 217 bp); |
| #2_C7orf10ex1-F/R | |
| (5′-GGCGAGAGACTCAGTGGATT-3′, | (SEQ ID No. 13) |
| 5′-ATCCCTTTAGCCACCCAGAC-3′, | (SEQ ID No. 14) |
| | and 403 bp); |
| #3_C7orf11-5upF/ex1R1 | |
| (listed above); | |
| #4_C7orf11ex1-F2/R3 | |
| (listed above); | |
| #5_C7orf11intron-F/R | |
| (5′-CGTTTGCCGAGCTCTTACTT-3′, | (SEQ ID No. 15) |
| 5′-GCAAGTTGGAAAACCACGTA-3′, | (SEQ ID No. 16) |
| | and 501 bp); |
| #6_C7orf11ex2-F/R2 | |
| (listed above); | |
| #7_C7orf11ex2-F3/R3 | |
| (5′-GGTTCAAGTCACAACTTTTAAGCA-3′, | (SEQ ID No. 17) |
| 5′-TCAAAGTCATCATCTTTGGGTAA-3′, | (SEQ ID No. 18) |
| | and 412 bp), |
| #8_3′ds-F1/R1 | |
| (5′-TGGCCATTTGGTTTGTTACC-3′, | (SEQ ID No. 19) |
| 5′-GCCCCTATAAGGAGACCCTCT-3′, | (SEQ ID No. 20) |
| | and 604 bp).; |
| #9_3′ds-F3/R3 | |
| (5′-CCACTCACACATCCATGTCC-3′, | (SEQ ID No. 21) |
| 5′-CAAACAAAAGCCAAAGCAAA-3′, | (SEQ ID No. 22) |
| | and 205 bp) |
| #10_3′ds-F4/R4 | |
| (5′-TCCTTTCTTGCAGGCTTGAT-3′, | (SEQ ID No. 23) |
| 5′-TTCTGAAAGAGCCAGCCAGT-3′, | (SEQ ID No. 24) |
| | and 305 bp), |
| #11_CDC2L5-3′UTR-F/R | |
| (5′-GAGTGAAGGCAGCCCTGTTA-3′, | (SEQ ID No. 25) |
| 5′-AAAAGGCAGAAGGCTGAGGT-3′, | (SEQ ID No. 26) |
| | and 300 bp). |

The numbers (#1-11) for the primer pairs correspond to those in FIG. 1(b). The primers used were DJg5/g6 (5′-AGCCAGGCCAGAGAACACTA-3 (SEQ ID No. 27)′ and 5′-GGGTCCTCCCTCTAGCCTTA-3′ (SEQ ID No. 28) that amplify a 704 bp fragment mapped on 7q36 as an internal control. The cycling conditions were: initial denaturation at 94° C. for 3 minutes, followed by 36 cycles of denaturation at 94° C. for 30 seconds, annealing at 58° C. for 30 seconds, and extension at 72° C. for 75 seconds.
Protein Sequences Encoded by Candidate Orthologues of the Human C7orf11 Gene

The accession numbers (NCBI or Ensembl (http://www.ensembl.org/)) of the protein sequences used for phylogenetic analysis (FIG. 2(a)) are NP_{—}619646 (human), ENSPTRP00000032652 (chimpanzee), BAB27916 (mouse), ENSRNOP00000018746 (rat), ENSGALP00000020100 (chicken), NP_{—}989025 (frog (Xenopus tropicalis)), CAF91712 (pufferfish (Tetraodon nigroviridis)), NP_{—}648690 (fruit fly (Drosophila melanogaster)), and XP_{—}318005 (mosquito (Anopheles gambiae)). Deduced protein sequences were derived from nucleotide acc. BC062385 (zebrafish (Danio rerio)) and BP456435 (pig). The dog protein sequence was predicted from the genomic DNA sequence (http://genome.ucsc.edu/). The evolutionary tree (FIG. 2(a)) was drawn by ClustalX (Thompson, J. D. et al. Nuc. Acids Res. 25, 4876-4882 (1997)).

Subcellular Localization Assay

A myc-tagged cDNA construct was generated for C7orf11 (mycC7orf11_wild) by inserting the coding region of C7orf11 (nt. 51-590 of NM_{—}138701) in pcDNA3.1+ myc his A (Invitrogen). A cDNA construct for the myc-tagged mutant C7orf11 protein (mycC7orf11_M144V) was also generated using QuikChange Site-Directed Mutagenesis Kit (Stratagene). The cDNA constructs were transfected in HEK293 (transformed human embryonic kidney cells), VA13 (SV40-transformed WI38 human embryo fibroblasts), or COS7 (SV40 transformed African green monkey kidney cells) using Lipofectamine-Plus (Invitrogen) and exposed to lipid-DNA complex in DMEM (Sigma-Aldrich) for 5 h. Two days after transfection, the cells were washed in PBS, and fixed in 50% Acetone plus 50% Methanol at −20° C. for 15 minutes. The samples were then blocked with 10% BSA in PBS (blocking buffer) at room temperature for 1 hour and incubated in blocking buffer containing anti-Myc antibody (diluted 1:50; Santa Cruz Biotechnology) at room temperature for 45 minutes in a chamber. Cells were washed three times with PBS, incubated with FITC-conjugated anti-mouse IgG antibody in blocking buffer (diluted 1:400; Santa Cruz Biotechnology) at room temperature for 30 minutes, and washed three times with PBS. DNA staining was performed by incubating in DAPI (0.05 μg/ml) at room temperature for 10 min and followed by rinse with 2×SSC. Coverslips were mounted in mounting medium (Vector). Fluorescent signals were analyzed by deconvolution microscopy (Zeiss).

In Situ RNA Hybridization

A 556 bp cDNA fragment corresponding to nt.303-858 of NM_{—}138701 was subcloned in pCR-Script (Stratagene) and used as template DNA to synthesize [S35]UTP-labeled riboprobes. Sense or antisense probes were hybridized with paraffin-embedded sections of developmental and adult skin tissues. The conditions used for hybridization, washing, signal detection were described previously (Saarialho-Kere et al, J. Clin. Invest. 90:1952-1957, (1992)).

Although preferred embodiments of the invention have been described herein in detail, it will be understood by those skilled in the art that variations may be made thereto without departing from the spirit of the invention.

TABLE 1
Genomic Sequence C7orf11 (Sequence ID No. 1)

chromosome: NCBI34 : 7 : 39912523 : 39915596 :-1

AGAGACTTCAGACCCTAATTGCTCTCCTCAGGGCGCCCGAGGAGGCAAGCTGGGGCAGGG
ATCCCTTTAGCCACCCAGACCGAATCTCGGAGGGTACCTGACTGCGGGCGGCCAGTCCAC
AGCCCCCTCCCGCCGCCCCGGCCGGAGAAGAGGCAGGTTCTGCGCAGAGCTGCCACCCTC
GCCAGCGTCGCCAGCATCGCGTGCGTCTCAGATGGCATCGGTCCCGGTGCAAGCGGCGAA
CTCCGTGCGGCGCCACCCGCCGCCACTGGGGGGCGCCCACGGACGCCGCAGACCCCGCCC
CCGCCGGGCGGGGCTGAGCACTAGTCGCCTGCTGGGCCAGCTTCTAGTCTGTCGGTCAGA
CCAAGGGCACTCAGACTGGAAAGGTTGTTGCTGGGCTTGGGTGTCCGTCCTTTCCTGTCT
CCTTTTCTTCCTCTTTTCACTTAAATCCACTGAGTCTCTCGCCAAGTCAGTGATTTAGGT
AATCCCGCCGCCCCTGGCGGAACCTTTGTGACGCCGGAACTGATGTGCCGTAGGGTGGCT
GCGGTCGGAGGACTGTTTTGGTAAAGGACTAGCAGTTCTCTGCGGAGGGCCGGTTGATAC
AGTTCCGGTGGGAGAACCCGGCTGCGAGGTTTTCGGCTTTGGCTCCTGATATGCAGCGAC
AGAATTTTCGGCCCCCAACTCCTCCTTACCCTGGTCCGGGTGGAGGAGGTTGGGGTAGCG
GAAGCAGCTTCCGCGGAACCCCGGGCGGGGGCGGACCACGGCCGCCCTCCCCTCGAGACG
GGTACGGGAGTCCGCACCACACGCCGCCGTACGGGCCCCGGTCTAGGCCGTACGGGAGCA
GTCACTCTCCGCGACACGGCGGCAGCTTCCCGGGGGGCCGGTTCGGGTCTCCGTCCCCTG
GCGGCTACCCTGGCTCCTACTCCAGGTCCCCCGCGGGGTCCCAGCAGCAATTCGGCTACT
CCCCAGGGCAGCAGCAGACCCACCCCCAGGTAATAATAATAGCCAGCGTTTGCCGAGCTC
TTACTTTATGGCAGGCACGGTACTAATATTCCCTTTACGTGGATTATTTTGTCTAATCCT
CTCTGAGGTACTATTGTTAGCCCAATTTTGCAGATGAGCGAACTGAGGCTTAGAGAAGTT
GAGTGACTTCCGGAAGATTATCCGAAAGAGTGGCAGAGCCGTACGTTCTGTTTTCGAGCC
CTCGTGCTTTTAACCAATACACTGCCACCCGTACACAGATTTTCCTAATTCTCCATTTCC
TCCTGTTTGCCAAAGGCCTTGCCAGTTTGTTTCAAGAAATAGTTCTTTTTCTTTTTTTTT
TTTTTTTTTTTATCTTTCTTTCGAGCTACACTGGCTTCTGCTCCAACTTCCCTGAAAAGA
AAATCGGTGGTAGTGGAAGTTTTCCATTCCTTTAGTGAGAGTAAATACAAAAGACAGAGA
GAGTATCTCTAGCATGTCTGGGATTTCTGTGGTTTACAACTTGCAACTACGTGGTTTTCC
AACTTGCTGTATTCTACACCAACCCCCTACCCCTTTTGAGCCTGGAGCTAAGGAGTAAAG
AAAATGTTCATGAGCTAGAAAGGTTCTAGAAACTTTAAAACTATAGTATTGTTTTCTCCT
GAAAAAAGTGAAAATTTCGAGGGAAAATTGAATATAATTACTTGATTTCATCACTGCTTG
TATTTAATTACTGCAAAGTTACTACATTAGACACAAAAGTATTTGAGATGACAGTGGTCA
TGGAATTATAAATAAATGAGAATAACGAATTGCTTTAAGTACTTTTTTTAAAAAATAGGA
ACACTTGATTAAAGGAAGCATGGACTTTGTAATTGAACATTATTTAGGTTCAAGTCACAA
CTTTTAAGCAGCAATGTGATTCCCGCTAACCTTTAGCTAAAGGAAGAAGTACCAATTATC
TTTACTAAGTGTTTTTTTAACTGATTTTTTTTCTAAAGGGTTCTCCAAGGACATCTACAC
CATTTGGATCAGGGCGTGTTAGAGAAAAAAGAATGTCTAATGAGTTGGAAAATTATTTCA
AGCCTTCAATGCTTGAAGATCCTTGGGCTGGCCTAGAACCAGTATCTGTAGTGGATATAA
GCCAACAATACAGCAATACTCAAACATTCACAGGCAAAAAAGGAAGATACTTTTGTTAAC
ATTTCTGAAATTCAACTGGAAGCTTCATGTGTCAGGAACATCTTGGACAAAACTTTAAGT
TGTGTTGATATAAATTTACCCAAAGATGATGACTTTGATTGGATAATTAGTAAGGTCTTT
TTGTTATTTTTCATCGTATCAGGTATTGTTGATATTAGAGAAAAAAGTAGGATAACTTGC
AACATTTAGCTCTGGAAGTACCTACCACATTTTAGAGATTTACCGTTTCCATATATTTAA
CATTCCTGGTTACATAATGGACATTTGTCTTTTAATGTTTTTTCAATGTTTTAAAATAAA
ACATTTTGTCTTCTAGCTATTGTGGTTTTGTGGTATGATAAAGAAGTAGACTTACTACAG
TAATGCTTTGTAGTCACTTAGAGTTCATAGGTAAATGTTTTGCAAATTATTTTTGAAAAT
GAAATAGGTAAACCATCCTTTGAGCTGTAGACAGCTCTGTATTTGTTCGTAAATAATGAG
GATGAGAATAACAGAGTTCTGTTCATACTCATTTTATCTAATGCTTCTGTTAAAATGCAT
TAAAACTTGAAGCCCCAGAAACCCATTTAAGCATAACTTTATACTCATTTAACTGGAAAA
GGCAAGAATAGACCATGCATTGCTAAATCAAAGTGTTCTATCTGAATTTCTCTATACCTC
TTAGCATTATGGAGTTAGTTTCTCTTGAGGCCAAAGAAGATAGCTACCAGCAGCCTCAAA
TCACTTCCATTGCAAAAAGAACATCTCTCATCTCCATCAATCCAAGAGAAGACAAATTTG
CCATACTTAGATCATGTTTCTGTATTATTGAACCAATTAACTGTGGCCAAGGCCACAGTG
AGACCTAATTAACTTGAATCATGGACACATCACCATAATGAGACAGGATTCTGTGACCTT
GAACACCACTGGGC

TABLE 2
cDNA C7orf11 Sequence (Sequence ID No. 2)

atgcagcgac agaattttcg gcccccaact cctccttacc ctggtccggg tggaggaggt 60
tggggtagcg gaagcagctt ccggggaacc ccgggcgggg gcggaccacg gccgccctcc 120
cctcgagacg ggtacgggag tccgcaccac acgccgccgt acgggccccg gtctaggccg 180
tacgggagca gtcactctcc gcgacacggc ggcagcttcc cggggggccg gttcgggtct 240
ccgtcccctg gcggctaccc tggctcctac tccaggtccc ccgcggggtc ccagcagcaa 300
ttcggctact ccccagggca gcagcagacc cacccccagg gttctccaag gacatctaca 360
ccatttggat cagggcgtgt tagagaaaaa agaatgtcta atgagttgga aaattatttc 420
aagccttcaa tgcttgaaga tccttgggct ggcctagaac cagtatctgt agtggatata 480
agccaacaat acagcaatac tcaaacattc acaggcaaaa aaggaagata cttttgttaa 540

TABLE 3
C7orf11 Protein Sequence (Sequence ID No. 3)

MQRQNFRPPTPPYPGPGGGGWGSGSSFRGTPGGGGPRPPSPRDGYGSPHHTPPY 179
GPRSRPYGSSHSPRHGGSFPGGRFGSPSPGGYPGSYSRSPAGSQQQFGYSPGQQ
QTHPQGSPRTSTPFGSGRVREKRMSNELENYFKPSMLEDPWAGLEPVSVVISQQ
YSNTQTFTGKKGRYFC

TABLE 5
Summary of the mutational analysis on the Amish brittle
hair syndrome (ABHS) patients for the candidate
genes in the 2 Mb candidate locus on chromosome 7p14

| | Accession | Exons | |
|---|---|---|---|
| Gene | number | sequenced* | Homozygous sequence variation |
| C7orf10 | NM_024728 | 15 exons | INV7-6T>C |
| | | | The same genotype was found in |
| | | | a control [symbol] |
| C7orf11 | NM_138701 | 2 exons | E2+91A>G (M144V), nt. 480 of |
| | | | NM_138701 Specific to ABHS |
| | | | patients |
| CDC2L5 | AJ297709 | 14 exons** | E1+194G>A (R21K), nt. 194 of |
| | | | AJ297709 |
| | | | The parents of the proband had |
| | | | the same genotype |
| RALA | NM_005402 | 4 exons | No variation |
| C7orf36 | NM_020192 | 3 exons | No variation |
| POU6F2 | NM_007252 | 10 exons | No variation |
| VPS41 | U87309 | Exon 1 to 10 | No variation |
| | | out of 29 | |
| | | exons | |

*Primer sequences and PCR conditions used are available upon request.
**The entire coding region and part of UTRs were sequenced

### REFERENCES

1. Price, V. H. et al. Arch. Derm. 116, 1375-1384 (1980).
2. Bergmann, E. & Egly, J. M. Trends Genet. 17, 279-286 (2001).
3. Botta E et al. Am J Hum Genet. 63, 1036-1048 (1998)
4. Weeda G, et al. Am J Hum Genet. 1997 60, 320-329 (1997)
5. Giglia-Mari, G. et al. Nat. Genet. 36, 714-719 (2004).
6. Botta, E. et al. Hum Mol Genet. 11, 2919-28 (2002)
7. Jackson, C. E. et al. Pediatrics 54, 201-207 (1974).
8. Baden, H. P. et al. Am. J. Hum. Genet. 28, 514-521 (1976).
9. Howell, R. R. et al. Am. J. Hum. Genet. 33, 957-967 (1981).
10. Pollitt, R. J. et al. Arch. Dis. Child. 43, 211-216 (1968).
11. Scherer, S. W. et al. Science 300, 767-772 (2003).
12. Nakabayashi, K et al. Genomics 79, 186-196 (2002).
13. Przedborski, S et al. Am. J. Med. Genet. 35, 566-573 (1990).
14. Rizzo R et al. J. Child. Neurol. 7, 300-303. (1992).

1. A nucleic acid sequence encoding a C7orf11 protein, said nucleic acid sequence having a mutation leading to the development of non-photosensitive Trichothiodystrophy (TTD).

2. The nucleic acid sequence of claim 1, wherein said sequence is represented by SEQ ID No. 1 or SEQ ID No. 2 having a mutation therein.

3. A fragment of the nucleic acid sequence of claim 2, wherein said fragment contains said mutation.

4. The fragment of claim 3, wherein said fragment is a probe or a primer.

5. The fragment of claim 4, wherein said fragment comprises 9 or more nucleotides.

6. The nucleic acid sequence of claim 2, wherein said mutation is a point mutation, deletion mutation, a frame-shift mutation or a null mutation.

7. The nucleic acid sequence of claim 3, wherein said mutation is a point mutation, deletion mutation, a frame-shift mutation or a null mutation.

8. The nucleic acid sequence of claim 6, wherein said point mutation encodes a M144V substitution in a C7orf11 gene product.

9. The nucleic acid sequence of claim 7, wherein said deletion mutation is a 2-bp deletion from exon 1 and said nucleic acid sequence encodes a 57 amino acid C7orf11 gene product.

10. The nucleic acid sequence of claim 7, wherein said deletion mutation is a deletion of a part of exon 1.

11. The nucleic acid sequence of claim 7, wherein said deletion mutation further comprises a deletion of exon 2.

12. The nucleic acid sequence of claim 7, wherein said null mutation is a deletion of exon 1 and exon 2.

13. A recombinant cloning vector comprising the nucleic acid molecule of claim 2.

14. The vector of claim 13, wherein said nucleic acid molecule is operatively linked to an expression control sequence that directs the expression of a coding sequence, said expression control sequence being selected from the group comprising sequences that control the expression of genes of prokaryotic and eukaryotic cells and their viruses and combinations thereof.

15. A host cell transformed with the vector of claim 14.

16. The host cell of claim 15 selected from the group consisting of E. Coli, Bacillus Subtilus, yeast, fungi, baculovirus, tobacco mosaic virus, plant and animal cells.

17. The primer of claim 4, selected from the group consisting of: SEQ ID No. 4 to SEQ ID No. 28.

18. A method for screening a subject to determine if said subject is a carrier of a mutant C7orf11 gene leading to the development or diagnosis of non-photosensitive TTD comprising:
determining from a biological sample from said subject the presence of a deletion mutation, point mutation, frame-shift mutation or a null mutation in the C7orf11 gene.

19. The method of claim 18, wherein the biological sample is blood.

20. A method of claim 18, wherein said method comprises:
isolating genomic DNA said biological sample from said subject;
hybridizing a DNA probe onto said isolated genomic DNA, said DNA probe spanning said mutation in said C7orf11 gene;
identifying said DNA probe with a suitable reagent for detecting the hybridization of said DNA probe to said C7orf11 gene having a mutation.

21. A method for screening a subject to determine if said subject is a carrier of a mutant form of the C7orf11 gene leading to the development or diagnosis of non-photosensitive TTD in said subject, said method comprising:
(a) assaying a biological sample of a subject to be screened, said sample containing a mutant or a normal C7orf11 gene, wherein the assay comprises:
(i) assaying for the presence of a normal C7orf11 gene by hybridization comprising: (A) an oligonucleotide probe which specifically binds to a normal nucleic acid sequence encoding a normal C7orf11 polypeptide, wherein the normal nucleic acid sequence comprises a nucleic acid sequence selected from the group consisting of:
(1) a nucleic acid sequence encoding a normal C7orf11 protein having the amino acid sequence shown in Table 3;
(2) a nucleic acid sequence which hybridizes under stringent conditions to at least 16 contiguous nucleotides of the nucleic acid sequence of (1); and
(3) a nucleic acid sequence complementary to the nucleic acid sequence of (1) or (2), and
(B) providing at least one reagent for detecting the hybridization of the oligonucleotide probe to said normal nucleic acid sequence; or
(ii) assaying for the presence of a mutant C7orf11 gene by hybridization comprising:
(A) an oligonucleotide probe which specifically binds to a mutant nucleic acid sequence encoding a mutant C7orf11 polypeptide, wherein the mutant nucleic acid sequence comprises a nucleic acid sequence selected from the group consisting of:
(1) a nucleic acid sequence encoding a mutant C7orf11 protein having a M144V amino acid substitution in the amino acid sequence depicted in Table 3;
(2) a nucleic acid sequence which hybridizes under stringent conditions to at least 16 contiguous nucleotides of the nucleic acid sequence of (1), said nucleic acid sequence containing said amino acid substitution; and
(3) a nucleic acid sequence complementary to the DNA sequence of (1) or (2); and
(B) providing at least one reagent for detecting the hybridization of the oligonucleotide probe to said mutant nucleic acid sequence,
wherein the probe and the reagent in (i) and (ii) are each present in amounts effective to perform the hybridization assay.

22. A kit for assaying the presence of a normal C7orf11 gene by hybridization, said kit comprising:
(a) an oligonucleotide probe which specifically binds to a normal nucleic acid sequence comprising a nucleic acid sequence selected from the group consisting of:
(i) a nucleic acid sequence encoding a normal C7orf11 protein having the amino acid sequence depicted in Table 3;
(ii) a nucleic acid sequence which hybridizes under stringent conditions to at least 16 contiguous nucleotides of the nucleic acid sequence of (i); and
(iii) a DNA sequence complementary to the nucleic acid sequence of (i) or (ii); and
(b) at least one reagent for detecting the hybridization of the oligonucleotide probe to said nucleic acid molecule, wherein the probe and the reagent are each present in amounts effective to perform the hybridization assay.

23. A kit for assaying the presence of a mutant C7orf11 gene by hybridization, said kit comprising:
(a) an oligonucleotide probe which specifically binds to a mutant nucleic acid sequence comprising a nucleic acid sequence selected from the group consisting of:
(i) a nucleic acid sequence encoding a mutant C7orf11 protein having an amino acid substitution M144V depicted in Table 3;
(ii) a nucleic acid sequence that contains a deletion in one or both of exons 1 and 2 as depicted in Table 2;
(iii) a nucleic acid sequence which hybridizes under stringent conditions to at least 16 contiguous nucleotides of the nucleic acid sequence of (i) or (ii), said nucleic acid sequence containing the amino acid substitution; and
(iv) a nucleic acid sequence complementary to the nucleic acid sequence of (i), (ii) or (iii); and
(b) at least one reagent for detecting the hybridization of the oligonucleotide probe to the DNA molecule,
wherein the probe and the reagent are each present in amounts effective to perform the hybridization assay.

24. The kit of claim 23, wherein said deletion is a 2 bp deletion in exon 1.

25. The kit of claim 23, wherein said deletion is a deletion of a portion of exon 1 and exon 2.

26. An anti-C7orf11 polyclonal or monoclonal antibody specific for a normal C7orf11 polypeptide of SEQ ID NO:3.

27. The polyclonal or monoclonal antibody of claim 26, wherein said polypeptide comprises at least one mutation, wherein said mutation is an amino acid substitution M144V in the sequence of SEQ ID NO: 3.

28. The polyclonal or monoclonal antibody of claim 26, wherein said polypeptide comprises a truncated 57 amino acid polypeptide.

29. A hybridoma producing a monoclonal antibody according to claim 27.

30. A method for identifying mutations in a sample C7orf11 comprising the steps of:
(a) quantitatively co-amplifying the exons of the sample C7orf11 gene using primers complementary to intron regions immediately flanking each of the exons;
(b) determining the lengths of the amplification products for each amplified sample exon and comparing that length to the length of amplification products obtained when a wild-type C7orf11 gene is amplified using the same primers, whereby differences in length between an amplified sample exon and the corresponding amplified wild-type exon reflect the occurrence of a deletion mutation in the sample C7orf11 gene.

31. A method for genetic screening of family members of an individual diagnosed as having non-photosensitive TTD, comprising the steps of:
(a) obtaining a patient blood sample from the diagnosed individual;
(b) quantitatively amplifying at least one exon of the C7orf11 gene in cells from the patient blood sample using primers complementary to intron regions immediately flanking each exon amplified; and
(c) determining the length of the amplification product for each exon amplified and comparing that length to the length of amplification products obtained when a wild-type C7orf11 gene is amplified using the same primers, whereby differences in length between an amplified sample exon and the corresponding amplified wild-type exon reflect the occurrence of an inherited deletion mutation in the C7orf11 gene of the diagnosed individual; and
(d) if an inherited mutation is identified, obtaining blood samples from the biological parents of the diagnosed individual; quantitatively amplifying the exon of the C7orf11 gene found to contain a deletion mutation in the patient blood sample in cells from the parent blood samples using the same primers used to amplify the exons in the patient blood sample; and determining the length of the amplification product for the exon in the amplified parent blood samples and comparing that length to the length of amplification products obtained when the patient blood sample was amplified, wherein in step (b) exons 1 and 2 are coamplified in a single reaction.

32. A method according to claim 31, wherein if at least one parent is found to carry the mutation found in the patient, further performing the steps of:
obtaining aunt/uncle blood samples from the siblings of the parent found to carry the mutation;
quantitatively amplifying the exon of the C7orf11 gene found to contain deletion mutation in the patient blood sample in cells from the aunt/uncle blood samples using the same primers used to amplify the exons in the patient blood sample; and determining the length of the amplification product for the exon in the amplified aunt/uncle blood samples and comparing that length to the length of amplification products obtained when the patient blood sample was amplified.

33. A kit for identification of mutations in the C7orf11 gene, comprising a plurality of primer pairs for multiplex co-amplification of exons 1 and 2 of the gene, wherein one member of each primer pair is labeled with a detectable label, and wherein the primer pairs are selected to have a common melting temperature and to produce amplification products having differing lengths and wherein the primers pairs bind to intron regions immediately flanking each of the selected plurality of exons, said kit comprising at least one set of primers for amplification of the selected plurality of exons of the C7orf11 gene or cDNA sequence.

34. A purified C7orf11 protein selected from a normal C7orf11 protein as shown in Table 3, or a mutant C7orf11 protein.

35. A fragment of the protein of claim 34, wherein said fragment is at least 8 amino acids.

36. The protein of claim 34, wherein said mutant protein has a M144V amino acid substitution in the amino acid sequence of Table 3.

37. The protein of claim 34, wherein said mutant protein is encoded by a cDNA shown in Table 2, wherein said cDNA sequence has a 2 bp deletion in exon 1.

38. The protein of claim 34, wherein said mutant protein is encoded by a cDNA shown in Table 2, wherein said cDNA sequence has a deletion of a part of exon 1.

39. The protein of claim 38, wherein said cDNA additionally has a deletion of exon 2.

40. A method of producing a mutant mammalian C7orf11 polypeptide comprising:
providing a cell transformed with a nucleic acid sequence encoding a mutant C7orf11 polypeptide positioned for expression in said cell;
culturing said transformed cell under conditions for expressing said nucleic acid; and
producing said mammalian C7orf11 polypeptide.

41. The method of claim 40, wherein the mutant C7orf11 polypeptide is encoded by a nucleic acid sequence set forth in Seq ID No. 1 or 2, wherein such nucleic acid sequence contains one or more mutations and/or deletions in the sequences contained therein.

42. The method of claim 41, wherein the mutant C7orf11 polypeptide contains one or more mutations and/or deletions in Seq ID No. 3.

43. A kit for assaying for the presence of a mutant C7orf11 gene by immunoassay techniques, said kit comprises:
(a) an antibody which specifically binds to a mutant gene product of the C7orf11 gene;
(b) reagent means for detecting the binding of the antibody to the gene product; and
(c) the antibody and reagent means each being present in amounts effective to perform the immunoassay.

44. A method for assessing the likelihood of developing non-photosensitive TTD in a fetal subject, said method comprising assaying for the presence of a mutant C7orf11 gene in said fetal subject by hybridization technique comprising:
(a) an oligonucleotide probe which specifically binds to a mutant C7orf11 gene;
(b) reagent means for detecting the hybridization of the oligonucleotide probe to the C7orf11 gene; and
(c) the probe and reagent means each being present in amounts effective to perform the hybridization assay.

45. The method of claim 44, wherein said method is used to assess the likelihood that said fetal subject will develop a symptom after birth selected from the group consisting of mental retardation, nail dystrophy, growth retardation, ichthyosis, decreased fertility, infertility and combinations thereof.

46. A method for treatment for non-photosensitive TTD in a patient, said method comprising the step of administering to the patient a therapeutically effective amount of the normal C7orf11 protein, or a functional fragment thereof, or a functional analogue thereof or a mimetic thereof.

47. A method of gene therapy for non-photosensitive TTD in a patient, said method comprising the delivery of a DNA molecule which includes a sequence corresponding to the normal DNA sequence encoding for normal C7orf11 protein.

48. A transgenic cell comprising a C7orf11 transgene having one or more mutations therein.

49. The transgenic animal of claim 48, wherein said animal exhibits one or more non-photosensitive TTD symptoms.
